(19) United States
(12) Patent Application Publication
(10) Pub. No.: US 2026/0357258 A1
(43) Pub. Date: Nov. 12, 2026

(54) Connector Modified Synthetic RIG-I Agonists and Methods of Using the Same

(71) Applicant: RIGImmune Inc., Farmington, CT (US)

(72) Inventor: Radhakrishnan P. IYER, Shrewsbury, MA (US)

(21) Appl. No.: 19/487,341

(22) PCT Filed: May 22, 2024
(86) PCT No.: PCT/US2024/030546

§ 371 Date: Nov. 24, 2025

Related U.S. Application Data
(60) Provisional application No. 63/468,612, filed on May 24, 2023.

(51) Int. Cl. C12N 15/113; A61K 31/7115; A61K 31/7125; A61K 47/54; A61P 31/14; A61P 31/16; A61P 37/04; C12N 15/117

(52) U.S. Cl. CPC C12N 15/113; A61K 31/7115; A61K 31/7125; A61K 47/549; A61P 31/14; A61P 31/16; A61P 37/04; C12N 15/117; C12N 2310/14; C12N 2310/17; C12N 2310/315; C12N 2310/317; C12N 2310/33; C12N 2310/3519; C12N 2310/531

(57) ABSTRACT

The present invention provides a composition comprising a nucleic acid compound capable of inducing interferon production wherein the nucleic acid compound comprises a first nucleic acid sequence and a second nucleic acid sequence, wherein the first nucleic acid sequence and the second nucleic acid sequence are complementary to each other and hybridize to form a double-stranded section of 8 or more base pairs and less than 20 base pairs; and wherein the two nucleic acid molecules are connected through a connector element that binds to a nucleotide of the first nucleic acid sequence and to a nucleotide of the second nucleic acid sequence.

### RELATED APPLICATION

This application claims the benefit of U.S. Provisional Application No. 63/468,612, filed on May 24, 2023. The entire teachings of the above application are incorporated herein by reference.

### BACKGROUND OF THE DISCLOSURE

RIG-I (retinoic acid-inducible gene I) is a cytosolic pattern recognition receptor (PRR) responsible for the type-1 interferon (IFN1) response. IFN1s have three main functions: to limit the virus from spreading to nearby cells, promote an innate immune response, including inflammatory responses, and help activate the adaptive immune system.

RIG-1 plays a key role in the innate immune system response to infection by a foreign organism, such as a bacterium or a virus. Exogenous nucleic acids, particularly viral nucleic acids, introduced into cells induce an innate immune response, resulting in, among other events, interferon (IFN) production and cell death. Upon sensing viral RNA, RIG-I-like receptors induce type I interferon (IFN) secretion leading to upregulation of antiviral IFN-induced proteins in the infected and neighboring cells, which inhibits virus replication. Further downstream events attract immune cells and trigger the adaptive immune response. In addition, RIG-I ligands have been reported to induce the apoptosis of many different types of tumor cells, but not of normal cells.

There remains a need for additional and improved compositions and methods to modulate the activity of immunomodulatory proteins. Such agents can be used for cancer immunotherapy and the prevention and treatment of other conditions, such as infections. Such agents can also be used as adjuvants for boosting the immunogenicity of vaccines against infectious diseases and cancer. There is therefore a need to develop improved RIG-I-like receptor ligands for diverse therapeutic immunomodulatory applications.

### SUMMARY OF THE DISCLOSURE

The present invention provides nucleic acid compounds capable of inducing interferon production. In one aspect, the nucleic acid compound comprises a first nucleic acid sequence and a second nucleic acid sequence, wherein the first nucleic acid sequence and the second nucleic acid sequence are complementary to each other and hybridize to form a double-stranded section, wherein the number of base pairs in the double stranded section is an integer ranging from 8 to 19; and wherein the 3′ end for the first nucleic acid sequence is linked to one end of a connector element and wherein the other end of the connector element is linked to the 5′ end of the second nucleic acid sequence, wherein element is as defined herein.

In embodiments, the RIG-I agonist has the structure of Formula I,

5′-P_{z}-(N)_{b}N-3′-(E)_{y}(E)-L-(E)(E)_{y′}-5′-N(N)_{b′}-3′
Formula I

wherein
5′-P_{z}—(N)_{b}N-3′ represents the first nucleic acid sequence;
5′-N(N)_{b′}-3′ represents the second nucleic acid sequence; P at each instance is independently a phosphate or analog thereof,
z is 2 or 3;
N is, at each instance, any nucleotide or modified nucleotide or analog or derivative there of;
b and b′ are independently 7, 8, 9, 10, 11, 12, 13, 14, 15, 16, 17, or 18;
5′-(E)_{y}(E)-L-(E)(E)_{y′}-3′ represents the connector element wherein
E at each occurrence is independently any nucleotide, modified nucleotide, or abasic;
y and y′ are independently 0-9, provided that y+y′ equals 0-8;
L is a non-nucleotide segment having the structure

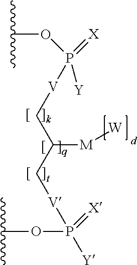

wherein
X and X′ are independently O or S;
Y and Y′ are independently OR″, SR″, or NRR′;
V and V′ are independently O, S, or NRR′;
q is 1-20;
k is 1-20;
t is 1-20;
M selected from aliphatic, substituted aliphatic, aryl, substituted aryl, heteroalkyl, heterocyclyl or substituted heterocyclyl;
W is any reactive group; and
d is 0 or 1.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 depicts the HPLC and Mass spec data of one embodiment of a biotin-conjugate according to the invention.

FIG. 2 depicts the HPLC and Mass spec data of one embodiment of a dye-conjugate according to the invention.

FIG. 3 depicts the stimulation of RIG-I by a compound according to the invention.

FIG. 4 provides illustrative examples of phosphomimics.

FIG. 5. depicts the activation of RIG-I in A549 cells.

FIG. 6 depicts the induction of the chemokine CXCL10.

### DETAILED DESCRIPTION

The present disclosure provides nucleic acid compounds that bind specifically to retinoic acid-inducible gene 1 receptor (RIG-I) and can activate the interferon response of RIG-I. In one aspect, the disclosure provides synthetic RNA molecules that agonize or activate one or more RIG-I. In certain embodiments, the disclosure provides compositions and methods for inducing the interferon response of RIG-I. In certain embodiments, the disclosure provides a nucleic acid compound. Exemplary nucleic acids for use in this disclosure include ribonucleic acids (RNA), deoxyribonucleic acids (DNAs), peptide nucleic acids (PNAs), threose nucleic acids (TNAs), glycol nucleic acids (GNAs), locked nucleic acids (LNAs) or a hybrid thereof. In certain embodiments, the nucleic acid is a ribonucleic acid (RNA).

As used herein, the terms “nucleic acid compound(s)” and “polynucleotide molecule(s)” can be used interchangeably to refer to a compound of Formula I or Formula II as described herein.

The present invention provides nucleic acid compounds (i.e., a RIG-I agonist) capable of inducing interferon production. In one aspect, the nucleic acid compound comprises a first nucleic acid sequence and a second nucleic acid sequence, wherein the first nucleic acid sequence and the second nucleic acid sequence are complementary to each other and hybridize to form a double-stranded section, wherein the number of base pairs in the double stranded section is an integer ranging from 8 to 19; and wherein the 3′ end for the first nucleotide sequence is conjugated to one end of a connector element and wherein the other end of the connector element is linked to the 5′ end of the second nucleotide sequence, wherein the connector element is as defined herein; and wherein the 5′ most nucleotide of the first nucleic acid sequence comprises a 5′ diphosphate or triphosphate moiety, or derivative or analog thereof. In embodiments, the nucleotides of the first and second nucleotide sequence are ribonucleic acids (RNA). The two strands need not be fully complementary by Watson Crick base pairing.

As used herein, the terms “first nucleic acid sequence”, “first nucleic acid molecule”, and “first nucleotide sequence” can be used interchangeably and the terms “second nucleic acid sequence”, “first nucleic acid molecule”, and “second nucleotide sequence” can be used interchangeably.

In embodiments, the RIG-I agonist has the structure of Formula I,

5′-P_{z}-(N)_{b}N-3′-(E)_{y}(E)-L-(E)(E)_{y′}-5′-N(N)_{b′}-3′
Formula I

wherein
5′-P_{z}—(N)_{b}N-3′ represents the first nucleic acid sequence;
5′-N(N)_{b′}-3′ represents the second nucleic acid sequence;
P at each instance is independently a phosphate or analog thereof,
z is 2 or 3;
N is, at each instance, any nucleotide or modified nucleotide or analog or derivative there of;
b and b′ are independently 7, 8, 9, 10, 11, 12, 13, 14, 15, 16, 17, or 18;
5′-(E)_{y}(E)-L-(E)(E)_{y′}-3′ represents the connector element wherein E at each occurrence is independently any nucleotide, modified nucleotide, or abasic;
y and y′ are independently 0-9, provided that y+y′ equals 0-8;
L is a non-nucleotide segment having the structure

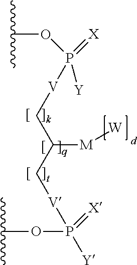

wherein
X and X′ are independently O or S;
Y and Y′ are independently OR″, SR″, or NRR′;
V and V′ are independently O, S, or NRR′;
q is 1-20;
k is 1-20;
t is 1-20;
M selected from aliphatic, substituted aliphatic, aryl, substituted aryl, heteroalkyl, heterocyclyl or substituted heterocyclyl;
W is any reactive group or conjugation group; and
d is 0 or 1.

The sequence of the first nucleic acid molecule, 5′-(N)_{b}N-3′, is complementary to the sequence of the second nucleic acid molecule, 3′-(N)_{b′}N-5′ and the first and second nucleic acid molecules hybridize to form a double stranded segment. N is, at each instance, any nucleotide or modified nucleotide or analog or derivative thereof, and b and b′ are independently 7, 8, 9, 10, 11, 12, 13, 14, 15, 16, 17, or 18 nucleotides in length. In embodiments, b and b′ are independently 9, 10, 11, 12, 13, 14, 15, 16, 17, or 18 nucleotides in length. In embodiments, b and b′ are independently 9, 10, 11, 12, 13, 14, or 15, nucleotides in length. In embodiments, b and b′ are independently 10, 11, 12, or 13 nucleotides in length.

In embodiments, b is 9 nucleotides in length and b′ is less than, equal to, or greater than 9 nucleotides in length. In embodiments, b is 10 nucleotides in length and b′ is less than, equal to, or greater than 10 nucleotides in length. In embodiments, b is 11 nucleotides in length and b′ is less than, equal to, or greater than 11 nucleotides in length. In embodiments, b is 12 nucleotides in length and b′ is less than, equal to, or greater than 12 nucleotides in length. In embodiments, b is 13 nucleotides in length and b′ is less than, equal to, or greater than 13 nucleotides in length. In embodiments, b is 14 nucleotides in length and b′ is less than, equal to, or greater than 14 nucleotides in length. In embodiments, b is 15 nucleotides in length and b′ is less than, equal to, or greater than 15 nucleotides in length. In embodiments, b is 16 nucleotides in length and b′ is less than, equal to, or greater than 16 nucleotides in length. In embodiments, b is 17 nucleotides in length and b′ is less than, equal to, or greater than 17 nucleotides in length. In embodiments, b is 18 nucleotides in length and b′ is less than, equal to, or greater than 18 nucleotides in length.

In embodiments, b=b′. In this embodiment, the nucleic acid compound comprises a blunt end. A blunt end refers to refers to, e.g., an RNA duplex where at least one end of the duplex lacks any overhang, e.g., a 3′-dinucleotide overhang, such that both the 5′- and 3′-strand end together, i.e., are flush or as referred to herein, are blunt. The molecules of the invention can have at least one blunt end. The molecules of the invention can have two blunt ends.

In embodiments, b does not equal b′. In this embodiment, the nucleic acid compound comprises an overhang. The term “overhang,” as used herein, refers to terminal non-base pairing nucleotide(s) resulting from one strand or region extending beyond the terminus of the complementary strand to which the first strand or region forms a duplex. One or more polynucleotides that are capable of forming a duplex through hydrogen bonding can have overhangs.

In certain embodiments, when b is less than b′, the nucleic acid compound has an overhang at the 3′ end of the second nucleic acid molecule. The single-stranded region extending beyond the 3′-end of the duplex is referred to as an overhang. In embodiments, the 3′-overhang comprises one non-base pairing nucleotide. In other embodiments, the 3′-overhang comprises two non-base pairing nucleotides.

In certain embodiments, when b is greater than b′, the nucleic acid molecule has a 5′-overhang. In other embodiments, the dsRNA structure produces a 5′-overhang. In yet other embodiments, the 5′-overhang comprises a non-base pairing nucleotide. In yet other embodiments, the 5′-overhang comprises two non-base pairing nucleotides.

In certain embodiments, the double-stranded section comprises one or more mispaired bases. That is, Watson-Crick base pairing is not required at each and every nucleotide pair.

In embodiments, the nucleic acid compound comprises a nucleotide insertion to create a kink in the double stranded region (see e.g., US 20210000856, which is incorporated herein by reference).

In embodiments, the nucleic acid compound can also contain internal bulge structure located in either the first nucleic acid sequence, the second nucleic acid sequence, or both.

As described herein, the nucleic acid compound of the disclosure is not dependent on a particular nucleotide sequence. Rather, any nucleotide sequence may be used, provided that the sequence has the ability to form the structure of a nucleic acid compound described herein.

Preferably, the first nucleic acid sequence and/or the second nucleic sequence are not antisense oligonucleotides and do not have antisense activity, i.e., the first nucleic acid sequence and/or the second nucleic sequence are not complementary to a (chosen) target nucleic acid sequence such that, when introduced into an animal or cell, the first nucleic acid sequence and/or the second nucleic sequence do not bind to and cause the reduction in the translation of RNA. Preferably, the first nucleic acid sequence and the second nucleic sequence do not have antisense activity.

In some aspects, the disclosure provides a nucleic acid compound wherein the nucleotide sequence comprising the compound is not complementary to a genomic DNA sequence or mRNA sequence, wherein the nucleic acid compound does not participate in RNA interference, and wherein the nucleic acid compound does not silence gene expression.

In certain embodiments, nuclease resistance of the nucleic acid compound can be enhanced with backbone modifications (e.g., phosphorothioates), sugar modifications and 5′-terminal modifications and/or 3′-terminal modifications.

In embodiments, the invention provides a polynucleotide molecule having the structure of Formula II,

5′-P_{z}-Nu-3′-(E)_{y}(E)-L-(E)(E)_{y′}-5′-Nu′-3′
Formula II

wherein
5′-P_{z}-Nu-3′ represents the first nucleic acid sequence;
5′-Nu′-3′ represents the second nucleic acid sequence;
P at each instance is independently a phosphate or analog thereof
z is 0, 1, 2, or 3;
5′-(E)_{y}(E)-L-(E)(E)_{y′}-3′ represents the connector element wherein
E at each occurrence is independently any nucleotide, modified nucleotide, or abasic;
y and y′ are independently 0-9, provided that y+y′ equals 0-8;
L is a non-nucleotide segment having the structure

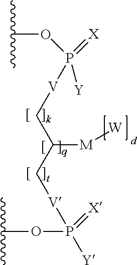

wherein
X and X′ are independently O or S;
Y and Y′ are independently OR″, SR″, or NRR′;
V and V′ are independently O, S, or NRR′;
q is 1-20;
k is 1-20;
t is 1-20;
M selected from aliphatic, substituted aliphatic, aryl, substituted aryl, heteroalkyl, heterocyclyl or substituted heterocyclyl;
W is any reactive group or conjugation group; and
d is 0 or 1.

In embodiments of Formula II, Nu and Nu′ are a sense strand and an antisense strand of an siRNA molecule. In embodiments, Nu is the sense strand and Nu′ is the antisense strand of an siRNA molecule. In embodiments, Nu is the antisense strand and Nu′ is the sense strand of an siRNA molecule.

In embodiments of Formula II, Nu is an antisense oligonucleotide and Nu′ is a nucleic acid sequence that is at least 80% complementary to the antisense oligonucleotide of Nu. In embodiments, the nucleic acid sequence of Nu′ is at least 90%, at least 93%, at least 95%, at least 975, at least 99%, or at least 100% complementary to the antisense oligonucleotide of Nu. In embodiments, the number of nucleotides of the nucleic acid sequence of Nu′ is the same as, less than, or greater than, the number of nucleotides of the antisense oligonucleotide of Nu.

In embodiments of Formula II, Nu′ is an antisense oligonucleotide and Nu is a nucleic acid sequence that is at least 80% complementary to the antisense oligonucleotide of Nu′. In embodiments, the nucleic acid sequence of Nu is at least 90%, at least 93%, at least 95%, at least 975, at least 99%, or at least 100% complementary to the antisense oligonucleotide of Nu′. In embodiments, the number of nucleotides of the nucleic acid sequence of Nu is the same as, less than, or greater than, the number of nucleotides of the antisense oligonucleotide of Nu′.

### The Connector Element

The connector element is a bivalent linker that connects the first nucleic acid sequence to the second nucleic acid sequence. The connector element comprises a 5′ nucleotide portion having 1 to 9 nucleotides, a non-nucleotide segment, and a 3′ nucleotide portion having 1 to 9 nucleotides, wherein the non-nucleotide segment has the structure L as defined herein, and wherein the total number of nucleotides in the 5′ nucleotide portion plus the 3′ nucleotide portion is 2 to 10 nucleotides. In embodiments, the nucleotides of the element are ribonucleic acid (RNA).

In embodiments, the connector element has the structure 5′-(E)_{y}(E)-L-(E)(E)_{y′}-3′:
wherein
E at each occurrence is independently any nucleotide, modified nucleotide, or abasic;
y and y′ are independently 0-7, provided that y+y′ equals 0-8;
L has the structure

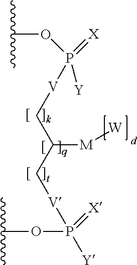

wherein
X and X′ are independently O or S;
Y and Y′ are independently OR″, SR″, or NRR′;
V and V′ are independently O, S, or NRR′;
q is 1-20;
k is 1-20;
t is 1-20;
M is selected from aliphatic, substituted aliphatic, aryl, substituted aryl, heterocyclyl or substituted heterocyclyl;
W is any reactive group or conjugation group; and
d is 0 or 1.

As discussed in further detail below, a targeting molecule (Tm) can be further attached to W, Y or Y′. Preferably, the Tm is attached to W.

In embodiments, q is 1, 2, 3, 4, 5, 6, 7, 8, 9, 10, 11, 12, 13, 14, 15, 16, 17, 18, 19, or 20. In embodiments, q is 1, 2, 3, 4, 5, 6, 7, 8, 9, 10, 11, 12, 13, 14, or 15. In embodiments, q is 1, 2, 3, 4, 5, 6, 7, 8, 9, or 10. In embodiments, q is 1, 2, 3, 4, or 5. In embodiments, q is 1. In embodiments, q is 2. In embodiments, q is 3. In embodiments, q is 4. In embodiments, q is 5.

In embodiments, k and t are independently 1, 2, 3, 4, 5, 6, 7, 8, 9, 10, 11, 12, 13, 14, 15, 16, 17, 18, 19, or 20. In embodiments, k and t are independently 1, 2, 3, 4, 5, 6, 7, 8, 9, 10, 11, 12, 13, 14, or 15. In embodiments, k and t are independently 1, 2, 3, 4, 5, 6, 7, 8, 9, or 10. In embodiments, k and t are independently 1, 2, 3, 4, or 5.

In embodiments, k is 1. In embodiments, k is 2. In embodiments, k is 3. In embodiments, k is 4. In embodiments, k is 5.

In embodiments, t is 1. In embodiments, t is 2. In embodiments, t is 3. In embodiments, t is 4. In embodiments, t is 5.

In embodiments, k and t are the same. In this embodiment, L is symmetrical.

In embodiments, k and t are different. In this embodiment, L is asymmetrical.

In embodiments, d is 0. In embodiments, d is 1.

In embodiments, R, R′, and R″ are independently selected from the group consisting of an alkyl, an amino alkyl, a carboxamido, polyethylene glycol (PEG), aralkyl, hetero-ar-alkyl, hetero-alkyl, substituted or unsubstituted cycloalkyl. In embodiments, R and R″ groups may contain functionalities such as amino, hydroxy, azido, or thiol, that can be optionally used for the attachment of which can be used to link to a targeting molecule (Tm), as described herein.

In certain embodiments, the R and R″ group can be a peptide group. Peptide groups include a variety of enzymatically cleavable or non-cleavable peptides. The individual amino acids groups of the peptide could be natural or synthetic amino acids.

In embodiments, R and R″ can be —CH2-O—CO—R¹, where R¹=Me, isopropyl, t-butyl, —(CH2)n-R², wherein R² is selected from aryl, aralkyl, heteroaryl, hetero-aralkyl. alkyl, an amino alkyl, a carboxamido, polyethylene glycol (PEG), hetero-alkyl, substituted or unsubstituted cycloalkyl. Suitable examples of R and R″ include, but are not limited to those shown below.

In certain embodiments, the reactive group W may be additionally connected to alkyl, an amino alkyl, a carboxamido, polyethylene glycol (PEG), aralkyl, hetero-ar-alkyl, hetero-alkyl, substituted or unsubstituted cycloalkyl. In embodiments, R and R″ groups may contain functionalities such as amino, hydroxy, azido, or thiol, that can be optionally used for the attachment to a targeting molecule (Tm).

In embodiments, M is selected from aliphatic, substituted aliphatic, aryl, substituted aryl, heteroralkyl, heterocyclyl or substituted heterocyclyl. The term “aliphatic group” or “aliphatic” refers to a non-aromatic moiety that may be saturated (e.g., single bond) or contain one or more units of unsaturation, e.g., double and/or triple bonds. An aliphatic group may be straight chained, branched or cyclic, contain carbon, hydrogen or, optionally, one or more heteroatoms and may be substituted or unsubstituted. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may include alkyl, substituted alkyl, alkenyl, substituted alkenyl, alkynyl, substituted alkynyl, and substituted or unsubstituted cycloalkyl groups as described herein.

The term “acyl” refers to a carbonyl substituted with hydrogen, alkyl, partially saturated or fully saturated cycloalkyl, partially saturated or fully saturated heterocycle, aryl, or heteroaryl. For example, acyl includes groups such as (C₁-C₆) alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” is intended to include both branched and straight chain, substituted or unsubstituted saturated aliphatic hydrocarbon radicals/groups having the specified number of carbons. Preferred alkyl groups comprise about 1 to about 24 carbon atoms (“C₁-C₂₄”). Other preferred alkyl groups comprise at about 1 to about 8 carbon atoms (“Ci-Cs”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means an aromatic system containing one, two or three rings wherein such rings may be attached together in a pendent manner or may be fused. The term “aryl” embraces aromatic radicals such as phenyl, naphthyl, tetrahydronaphthyl, indane furanyl, quinazolinyl, pyridyl and biphenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g., pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g., morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g., 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g., benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl” refers to heterocyclo-substituted alkyl radicals. More preferred heterocycloalkyl radicals are “lower heterocycloalkyl” radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are “lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms “aralkyl” or “arylalkyl” refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals. Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g., CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The terms “halogen” or “halo” as used herein, refers to an atom selected from fluorine, chlorine, bromine and iodine.

Suitable M groups include, but are not limited to, substituted or unsubstituted C₁ to C₁₅ alkyl, substituted or unsubstituted C₂-C₁₆-alkenylene, or substituted or unsubstituted C₂-C₁₆-alkynylene, aralkyl, hetero-ar-alkyl, hetero-alkyl, substituted or unsubstituted cycloalkyl.

In embodiments, M is optionally substituted W, wherein W is defined herein. In embodiments, M is substituted W.

In embodiments, M can comprise a non-nucleotide linker selected from the group consisting of:
(a) an ethylene glycol linker; and
(b) an alkyl linker.

In some embodiments, M is a hexaethylene glycol linker. In some embodiments, M is a C9 alkyl linker.

Non-limiting examples of M include ethylene glycols (—CH₂CH₂O), peptides, peptide nucleic acids (PNAs), alkylene chains (a divalent alkane-based group), amides, esters, ethers, and so forth, and any combinations thereof.

In certain embodiments, M comprises at least one ethylene glycol group. In other embodiments, M comprises one ethylene glycol group. In yet other embodiments, M comprises two ethylene glycol groups. In yet other embodiments, M comprises three ethylene glycol groups. In yet other embodiments, M comprises four ethylene glycol groups. In yet other embodiments, M comprises five ethylene glycol groups. In yet other embodiments, M comprises six ethylene glycol groups. In yet other embodiments, M comprises seven ethylene glycol groups. In yet other embodiments, M comprises eight ethylene glycol groups. In yet other embodiments, M comprises nine ethylene glycol groups. In yet other embodiments, M comprises ten ethylene glycol groups. In yet other embodiments, M comprises more than ten ethylene glycol groups. In yet other embodiments, M comprises (OCH₂CH₂)n, wherein n is an integer ranging from 1 to 10. In yet other embodiments, n is 1. In yet other embodiments, n is 2. In yet other embodiments, n is 3. In yet other embodiments, n is 4. In yet other embodiments, n is 5. In yet other embodiments, n is 6. In yet other embodiments, n is 7. In yet other embodiments, n is 8. In yet other embodiments, n is 9. In yet other embodiments, n is 10.

In certain embodiments, M comprises at least one amino acid, at least two amino acids, at least three amino acids, at least four amino acids, at least five amino acids, at least six amino acids, at least seven amino acids, at least eight amino acids, at least nine amino acids, at least ten amino acids, or more than ten amino acids.

In certain embodiments, M comprises a alkyl or alkylene chain, such as but not limited to a C₁-C₅₀ alkyl or alkylene chain, which is optionally substituted with at least one substituent selected from the group consisting of C₁-C₆ alkyl, C₁-C₆ haloalkyl, C₁-C₆ alkyl, C₃-C₈ cycloalkyl, C₁-C₆ alkoxy, —OH, halo, —NH₂, —NH(C₁-C₆ alkyl), —N(C₁-C₆ alkyl)(C₁-C₆ alkyl), —C(═O)OH, —C(═O)O(C₁-C₆ alkyl), and —C(═O)O(C₃-C₅ cycloalkyl), wherein the alkyl or cycloalkyl is optionally substituted with at least one selected from the group consisting of C₁-C₆ alkyl, C₁-C₆ haloalkyl, C₁-C₆ alkyl, C₃-C₈ cycloalkyl, C₁-C₆ alkoxy, —OH, halo, —NH₂, —NH(C₁-C₆ alkyl), —N(C₁-C₆ alkyl)(C₁-C₆ alkyl), —C(═O)OH, —C(═O)O(C₁-C₆ alkyl), and —C(═O)O(C₃-C₈ cycloalkyl). In other embodiments, M is selected from the group consisting of —(CH₂)—, —(CH₂)₂—, —(CH₂)₃—, —(CH₂)₂—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—, —(CH₂)₁₆—, —(CH₂)₁₇—, —(CH₂)₁₈—, —(CH₂)₁₉—, and —(CH₂)₂₀—, each of each is independently optionally substituted.

The non-nucleotide segment binds to the 5′ nucleotide portion of the element and 3′ nucleotide portion of the element, at either the 3′ position of the sugar or at the phosphorous-containing internucleotide linkage. In embodiments, the non-nucleotide segment binds to the 5′ nucleotide portion of the element and 3′ nucleotide portion of the element at the 3′ position of the sugar. In embodiments, the non-nucleotide segment binds to the 5′ nucleotide portion of the element and 3′ nucleotide portion of the element at the phosphorous-containing internucleotide linkage. In embodiments, the non-nucleotide segment binds to the 3′ position of the sugar of a nucleotide on the 5′ or 3′ nucleotide portion of the element to the phosphorous-containing internucleotide linkage of the other nucleotide portion of the element.

Oligonucleotide synthesis starts with the 3-end of the sequence which is the bottom strand (the sequence that does not have a di or triphosphate group). Thus, the end of the sequence will have a “free” 5′-OH group that will be then linked to the connector via a phosphate group. The connector segment is linked to the 5′-OH group of the element through a phosphate group. The other end of the connector has a hydroxy group that will be linked to the 3′-OH group of the terminal nucleotide of the upper strand (or sequence that has the 5′-DP or TP group) through a phosphate group. (see e.g., the Scheme for synthesis of Compound 1).

In Canonical oligonucleotides, the internucleotide connectivity is through 3′ hydroxy of one nucleotide with the 5′OH of the second nucleotide through phosphate linkage. In non-canonical oligonucleotides, the connectivity is between the 2′ hydroxy of one nucleotide with 5′-end of the second nucleotide.

In some embodiments, the non-nucleotide segment is bound symmetrically to the 5′ nucleotide portion of the element and the 3′ nucleotide portion of the element. “Symmetrically” refers to an element wherein y=y′ such that the nucleotides of the 5′ nucleotide portion of the element and 3′ nucleotide portion of the element are bound to the non-nucleotide segment at the same distance (in terms of the number of nucleotides) from the 3′ end of the first nucleic acid sequence as it is from the 5′ end of the second nucleic acid sequence. Preferably, y and y′ are 0 to 7. Preferably, y and y′ are 0 to 4. Preferably, y and y′ are 0. Preferably, y and y′ are 1. Preferably, y and y′ are 2. Preferably, y and y′ are 3. Preferably, y and y′ are 4. Preferably, y and y′ are 5. Preferably, y and y′ are 6. Preferably, y and y′ are 7. Non-limiting examples of a symmetrical connector element that binds the first nucleotide sequence and to the second nucleotide sequence of the RIG-I agonist are as follows:

5′-E-L-E-3′
5′-EE-L-EE-3′;
5′-EEE-L-EEE-3′;
5′-EEEE-L-EEEE-3′;
or
5′-EEEEE-L-EEEEE-3′.

In some embodiments, the non-nucleotide segment is bound asymmetrically to the 5′ nucleotide portion of the element and the 3′ nucleotide portion of the element. “Asymmetrically” refers to an element where y does not equal y′ such that the number of nucleotides in the 5′ nucleotide portion of the element is not the same as the number of nucleotides in the 3′ nucleotide portion of the element. For example, when y is 0, y′ is 1, 2, 3, 4, 5, 6, 7, 8 or 9. Alternatively, for example, when y′ is 0, y is 1, 2, 3, 4, 5, 6, 7, 8 or 9. Non-limiting examples of asymmetrical connector elements that binds the first nucleotide sequence and to the second nucleotide sequence of the RIG-I agonist are as follows:

| | |
|---|---|
| 5′-E-L-EE-3′ | 5′-EE-L-E-3′ |
| 5′-E-L-EEE-3′ | 5′-EEE-L-E-3′ |
| 5′-E-L-EEEE-3′ | 5′-EEEE-L-E-3′ |
| 5′-E-L-EEEEE-3′ | 5′-EEEEE-L-E-3′ |
| 5′-E-L-EEEEEE-3′ | 5′-EEEEEE-L-E-3′ |
| 5′-E-L-EEEEEEE-3′ | 5′-EEEEEEE-L-E-3′ |
| 5′-E-L-EEEEEEEE-3′ | 5′-EEEEEEEE-L-E-3′ |
| 5′-E-L-EEEEEEEEE-3′ | 5′-EEEEEEEEE-L-E-3′ |
| 5′-EE-L-EEE-3′; | 5′-EEE-L-EE-3′ |
| 5′-EE-L-EEEE-3′ | 5′-EEEE-L-EE-3′ |
| 5′-EE-L-EEEEE-3′ | 5′-EEEEE-L-EE-3′ |
| 5′-EE-L-EEEEEE-3′ | 5′-EEEEEE-L-EE-3′ |
| 5′-EE-L-EEEEEEE-3′ | 5′-EEEEEEE-L-EE-3′ |
| 5′-EE-L-EEEEEEEE-3′ | 5′-EEEEEEEE-L-EE-3′ |
| 5′-EEE-L-EEEE-3′; | 5′-EEEE-L-EEE-3′; |
| 5′-EEE-L-EEEEE-3′; | 5′-EEEEE-L-EEE-3′; |
| 5′-EEE-L-EEEEEE-3′; | 5′-EEEEEE-L-EEE-3′; |
| 5′-EEE-L-EEEEEEE-3′; | 5′-EEEEEEE-L-EEE-3′; |
| 5′-EEEE-L-EEEEE-3′; or | 5′-EEEEE-L-EEEE-3′. |

### Conjugation Group (W)

W is any reactive group or conjugation group which can be used to attach a variety of small and large targeting molecules (Tm) to the nucleic acid compound of the invention. In embodiments, W is selected from the group consisting of an alkyl, an amino alkyl, a carboxamido, polyethylene glycol (PEG), aralkyl, hetero-ar-alkyl, hetero-alkyl, substituted or unsubstituted cycloalkyl. The W may contain functionalities such as amino, hydroxy, azido, or thiol, that can be used for the attachment to a targeting molecule (Tm).

In embodiments, W is a reactive group selected from OR, NRR′, SR, or N3, wherein R and R′ are as defined herein.

In embodiments, the W can be a peptide group. Peptide groups include a variety of enzymatically cleavable or non-cleavable peptides. The individual amino acids groups of the peptide could be natural or synthetic amino acids. In embodiments, W can be —CH₂—O—CO—R₁, where R₁=Me, isopropyl, t-butyl, —(CH₂)n-R₂, wherein R₂ is selected from aryl, aralkyl, heteroaryl, hetero-aralkyl. alkyl, an amino alkyl, a carboxamido, polyethylene glycol (PEG), hetero-alkyl, substituted or unsubstituted cycloalkyl. Suitable examples of W include, but are not limited to those shown below.

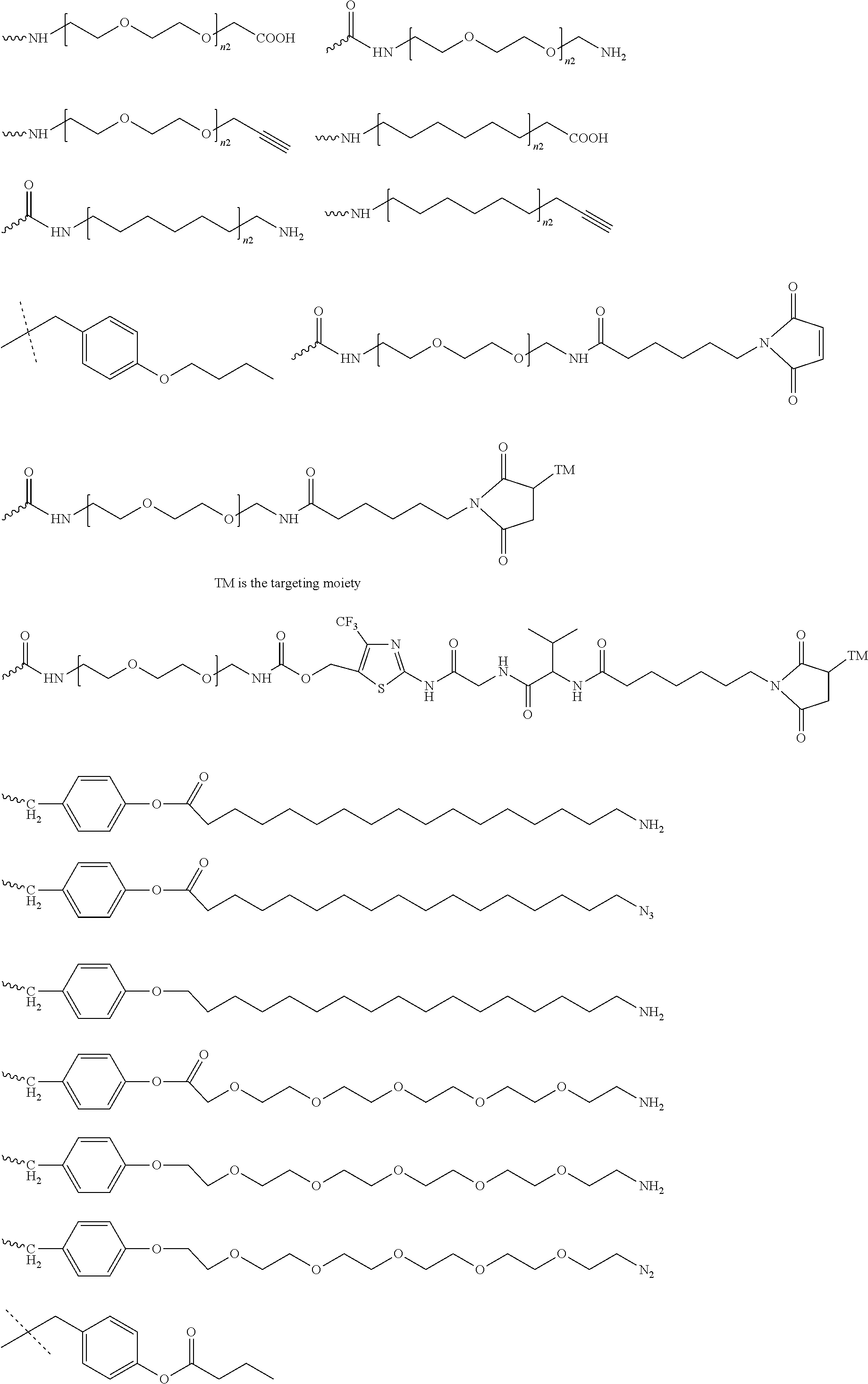

W can be a bifunctional connector wherein the bifunctional connector groups can be of different compositions which enables connecting the two ends of the nucleic acid strands. Non-limiting examples include the following:

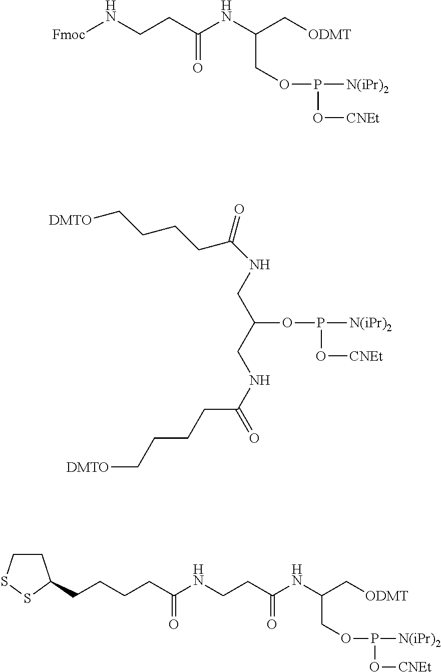

### Targeting Molecule (Tm)

In embodiments of the invention, targeting molecules (Tm) can be conjugated to the element via W or Y or Y′. In embodiments, targeting molecules (Tm) can be conjugated to the element via W. In embodiments, targeting molecules (Tm) can be conjugated to the element via Y. In embodiments, targeting molecules (Tm) can be conjugated to the element via Y′.

Tm can include, without limitation, molecules such as vitamins, biotin, folic acid, peptides, Vitamin D, antibodies and proteins such as integrins, fatty acids and esters, cell-penetrating peptides, and tissue and cell-targeting agents such as N-acetyl glucosamine. In embodiments, Tm can include such groups as a targeting antibody or targeting moiety. In embodiments, Tm can include such groups as fluorescent dyes.

In some embodiments, Tm is an antibody, a hormone, a hormone derivative, folic acid, a folic acid derivative, a biotin, a small molecule, an oligopeptide, a sigma-2-ligand, or a sugar, fatty acid, ionic, non-ionic or ionizable lipids.

In some cases, Tm could be a dendrimer based on glycol, alkyl diamine core structure.

In some embodiments, the antibody is selected from intact polyclonal antibodies, intact monoclonal antibodies, antibody fragments, single chain Fv (scFv) mutants, multispecific antibodies, bispecific antibodies, chimeric antibodies, humanized antibodies, human antibodies, fusion proteins including an antigen determination portion of an antibody, and other modified immunoglobulin molecules including an antigen recognition site.

In some embodiments, the antibody is selected from muromonab-CD3, abciximab, rituximab, daclizumab, palivizumab, infliximab, trastuzumab, etanercept, basiliximab, gemtuzumab ozogamicin, alemtuzumab, ibritumomab tiuxetan, adalimumab, alefacept, omalizumab, efalizumab, tositumomab-I¹³¹, cetuximab, bevacizumab, natalizumab, ranibizumab, panitumumab, eculizumab, rilonacept, certolizumab pegol, romiplostim, belimumab, anti-CD20, tocilizumab, atlizumab, mepolizumab, pertuzumab, tremelimumab, ticilimumab, inotuzumab ozogamicin, aflibercept, catumaxomab, pregovomab, motavizumab, efumgumab, Aurograb®, raxibacumab, and veltuzumab. In some embodiments, the antibody is selected from an anti-CD22 antibody or an anti-CD79b antibody.

In some embodiments, the hormone is a steroid. In some embodiments, the hormone is selected from estrogen, testosterone, dihydrotestosterone, and ethisterone.

In some embodiments, Tm is a sterol. In some embodiments, Tm is cholesterol, beta-sitosterol, phytosterols or any derivative thereof.

In some embodiments, Tm is folic acid or any derivative thereof. In some embodiments, Tm is biotin. In some embodiments, Tm is a substituted benzodiazepine. In some embodiments, Tm is a glutamate-urea-lysine. In some embodiments, Tm is asparaginyl-glycinyl-aginine oligopeptide.

In some embodiments, Tm is an integrin ligand. In some embodiments, the integrin ligand is an RGD peptide. In some embodiments, the RGD peptide is an Arg-Gly-Asp oligopeptide.

In some embodiments, Tm is a sigma-2-ligand.

In embodiments, Tm is a lipid. In embodiments, Tm is an ionizable lipid. In embodiments, Tm is a cationic lipid.

In embodiments, Tm is polyethylene glycol (PEG), 1,2-Dimyristoyl-sn-glycero-3-methoxypolyethylene glycol (PEG-DMG), 9-Heptadecanyl 8-{(2-hydroxyethyl)[6-oxo-6-(undecyloxy)hexyl]amino}octanoate (e.g., amino lipid SM-102) or any derivative thereof.

In embodiments, Tm is 1,2-Di-(9Z-octadecenoyl)-3-trimethylammonium propane methylsulfate (DOTAP), Distearoylphosphatidylcholine (DSPC), or any derivative thereof.

In some embodiments, Tm is oleic acid or a pharmaceutically acceptable salt thereof.

In some embodiments, Tm is a sugar. In some embodiments, the sugar is galactose. In some embodiments, the sugar is N-acetyl-galactosamine.

### 5′ Terminal Phosphate (P)

The nucleic acid compound of the invention comprises a 5′-diphosphate ((HO)2(O)P—O—P(HO)(O)—O-5′); 5′-triphosphate ((HO)2(O)P—O—(HO)(O)P—O—P(HO)(O)—O-5′) or includes a phosphoryl analog at the 5′ terminus. The presence of a 5′ triphosphate or 5′ diphosphate, or analogs thereof, may improve the binding affinity of the nucleic acid molecule.

Suitable analogs include: 5′-guanosine cap (7-methylated or non-methylated) (7m-G-O-5′-(HO)(O)P—O—(HO)(O)P—O—P(HO)(O)—O-5′); 5′-adenosine cap (Appp), any modified or unmodified nucleotide cap structure (N—O-5′-(HO)(O)P—O—(HO)(O)P—O—P(HO)(O)—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-).

In other examples, the terminal group could be-(HO)(O)P—X—P(HO)(Y)—O-5′), where X and Y could be independently, S, NR, O; where R could be C1-C20 alkyl, aralkyl.

Other terminal groups may include mixed carboxy-phosphoryl, sulfonyl phosphoryl, or other phosphomimics known in the art. For example, (OH—CO—O—P(O)—(OH)—O—P(O)(OH)—O-5′), OH—S(O)(O)—O—P(O)—(OH)—O—P(O)(OH)—O-5′), (OH—S(O)(O)—O—P(O)(OH)—O-5′), (OH—CO—CH₂—P(O)—O—CH₂—, OH—S(O)(O)—CH₂—P(O)(OH)—O-5′).

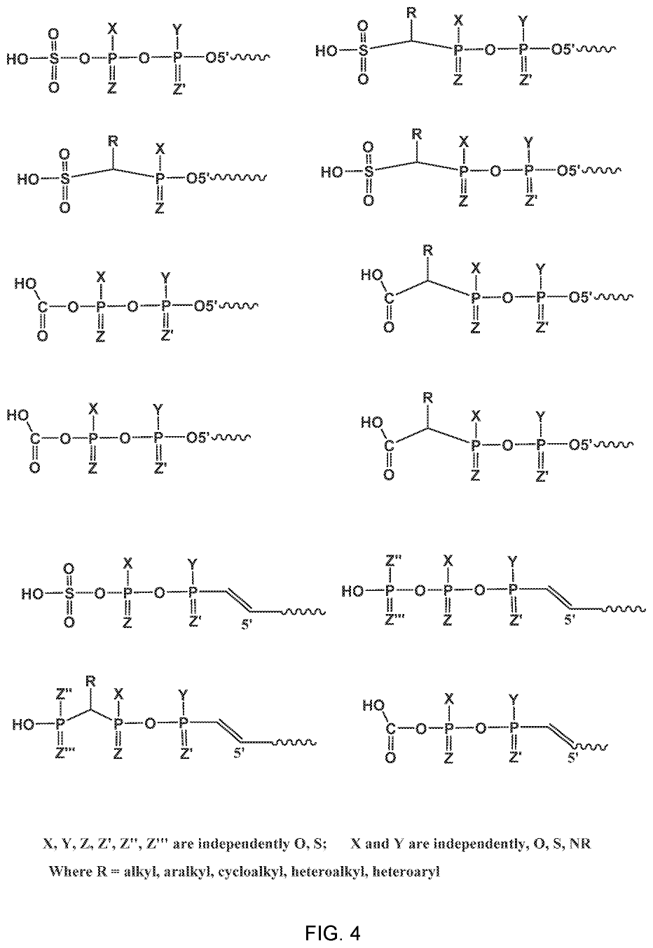

Illustrative examples of phosphomimics are shown in FIG. 4.

It is to be noted that a single internucleotidic phosphorothioate linkage in the composition can exist in two isomeric forms designated as Rp and Sp. Compounds disclosed in the invention can be individual isomeric forms or mixed Rp,Sp compositions. Other linkages can exist in isomeric or diastereomeric forms.

In one embodiment, the nucleic acid molecule comprises a 5′ triphosphate wherein the phosphates are unmodified. In one embodiment, the nucleic acid molecule comprises a 5′ triphosphate wherein at least one of the phosphates is a phosphate analog. In one embodiment, the nucleic acid molecule comprises a 5′ triphosphate wherein two of the phosphates are a phosphate analog. In one embodiment, the nucleic acid molecule comprises a 5′ triphosphate wherein all three of the phosphates are a phosphate analog.

In one embodiment, the nucleic acid molecule comprises a 5′ diphosphate wherein the phosphates are unmodified. In one embodiment, the nucleic acid molecule comprises a 5′ diphosphate wherein at least one of the phosphates is a phosphate analog. In one embodiment, the nucleic acid molecule comprises a 5′ diphosphate wherein both of the phosphates are a phosphate analog.

In embodiments, the phosphate analog comprises the structure

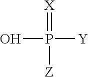

wherein
Y is O or S, or CH—R where R=alkyl, ar-alkyl, heteroaryl, cycloalkylamines (e.g., piperazines),
X is O or S, and
Z is OH, SH, NHR′, wherein R′ is H, alkyl, aralkyl, or heteroaryl.

In embodiments, the nucleic acid compound comprises a 5′ diphosphate comprising the structure:

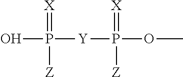

wherein
Y is O or S, or CH—R where R=alkyl, ar-alkyl, heteroaryl, cycloalkylamines (e.g., piperazines),
X is independently O or S, and
Z is independently OH, SH, NHR′, wherein R′ is H, alkyl, aralkyl, heteroaryl.

In embodiments, the nucleic acid compound comprises a 5′ triphosphate comprising the structure:

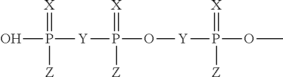

wherein
Y is independently O or S, or CH—R where R=alkyl, ar-alkyl, heteroaryl, cycloalkylamines (e.g., piperazines),
X is independently O or S, and
Z is independently OH, SH, NHR′, wherein R′ is H, alkyl, aralkyl, or heteroaryl.

### Nucleotide and Modifications

In embodiments, any nucleotide or abasic within the first nucleotide sequence, the second nucleotide sequence and/or within the connector element independently comprises naturally occurring nucleobase or a modified nucleobase, a naturally occurring internucleoside linkage or a modified internucleoside linkage, naturally occurring sugar or a modified sugar, or combinations thereof.

In embodiments, any nucleotide or abasic within the first nucleotide sequence, the second nucleotide sequence and/or within the connector element independently comprises naturally occurring nucleobase or a modified nucleobase.

As used herein, “nucleic acid”, “oligonucleotide”, “nucleotide sequence”, or “nucleotide portion”, can be used interchangeably and generally refer to a molecule or compound comprising a plurality of linked nucleosides. In certain embodiments, a nucleic acid comprises one or more modified or unmodified ribonucleosides (RNA) and/or one or more modified or unmodified deoxyribonucleosides (DNA). In certain embodiments, a nucleic acid comprises one or more modified or unmodified ribonucleosides (RNA). In certain embodiments, a nucleic acid consists of one or more modified or unmodified ribonucleosides (RNA).

The term “ribonucleotide” and the phrase “ribonucleic acid” (RNA), as used herein, refer to a modified or unmodified nucleotide or polynucleotide comprising at least one ribonucleotide unit. A ribonucleotide unit comprises an oxygen attached to the 2′-position of a ribosyl moiety having a nitrogenous base attached in N-glycosidic linkage at the 1′-position of a ribosyl moiety, and a moiety that either allows for linkage to another nucleotide or precludes linkage.

In certain embodiments, a nucleic acid comprises unmodified ribonucleosides (RNA). In certain embodiments, a nucleic acid comprises one or more modified ribonucleosides.

As used herein, “modified nucleic acid” means a nucleic acid molecule or compound comprising at least one modified nucleoside and/or at least one modified sugar.

The term “nucleoside” generally refers to compounds consisting of a sugar, usually ribose, deoxyribose, pentose, arabinose or hexose, and a purine or pyrimidine base. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “nucleotide” generally refers to a nucleoside comprising a phosphorous-containing group attached to the sugar. As used herein, “linked nucleosides” may or may not be linked by phosphate linkages and thus includes, but is not limited to, “linked nucleotides.” As used herein, “linked nucleosides” are nucleosides that are connected in a continuous sequence (i.e., no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified. As used herein, “nucleobase sequence” means the order of contiguous nucleobases independent of any sugar, linkage, or nucleobase modification.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C or 6-methyl C), and uracil (U).

As used herein, “modified nucleobase” means any nucleobase that is not a naturally occurring nucleobase. Exemplary modified nucleobases include, but are not limited to, 7-deazaadenine, 7-deazaguanine, 7-deaza-8-azaguanine, 7-deaza-8-azaadenine, N6 delta 2-isopentenyladenine (6iA), N6-delta 2-isopentenyl-2-methylthioadenine (2 ms6iA), N2-dimethylguanine (dmG), 7methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, 06-methylguanine, N6-methyladenine, 04-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

As used herein, “modified nucleoside” means a nucleoside comprising at least one chemical modification compared to naturally occurring RNA or DNA nucleosides. Modified nucleosides comprise a modified sugar moiety and/or a modified nucleobase.

In other embodiments, the nucleobase can be a “universal” nucleobase such as without limitation nitro-substituted aromatic molecules such as 3-nitro pyrrole, 4-nitropyrazole, 4-nitro-imidazole, 5-nitro indole.

In other embodiments, the nucleobase can be a unnatural hydrophobic pyrimidine-like N-nucleosides or unnatural hydrophobic N-nucleosides such as Isocarbostyril, 3-methylisocarbostyril, 5-methylisocarbostyril, 3,5-dimethyl-2-pyridone, 7-Propynyl-3-methylisocarbostyril, 7-propynylisocarbostyril, 7-propynyl-3-methyl-2(1H), or unnatural hydrophobic purine-like nucleosides such as 7-azaindole, 6-methyl-7-azaindole, imidazole pyridine, 3-propynyl-7-azaindole, 3-propynyl-4,7-diazaindole.

In other embodiments, the nucleobase can be C-nucleosides such as 3,5-dimethylphenyl-C-nucleoside, 1,4-dimethylnaphthalene-C-nucleoside or other C-nucleosides derived from Trimethylbenzene, dimethylbenzene, dimethylnaphthalene, 3-methyl-2-naphthalene, 1-methyl-3-naphthalene, or 2-naphthalene.

### Internucleoside Linkage

In embodiments, any nucleotide or abasic within the first nucleotide sequence, the second nucleotide sequence and/or within the connector element independently comprises naturally occurring internucleoside linkage or a modified internucleoside linkage.

As used herein “internucleoside linkage” means a covalent linkage between adjacent nucleosides in an oligonucleotide. As used herein “naturally occurring internucleoside linkage” means a 3′ to 5′ phosphodiester linkage. As used herein, “modified internucleoside linkage” means any internucleoside linkage other than a naturally occurring internucleoside linkage. The nucleoside residues of the nucleic acid compound of the invention can be coupled to each other by any of the numerous known internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Methods of preparation of phosphorous-containing and non-phosphorous-containing internucleoside linkages are well known to those skilled in the art.

Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. In some embodiments, the nucleic acid compound of the invention may comprise combinations of internucleotide linkages. In some embodiments, the nucleic acid compound of the invention may comprise combinations of phosphorothioate and phosphodiester internucleotide linkages. In some embodiments more than half but less that all of the internucleotide linkages are phosphorothioate internucleotide linkages. In some embodiments all of the internucleotide linkages are phosphorothioate internucleotide linkages.

In embodiments, the nucleic acid compound comprises one or more peptide nucleic acids (PNA). A peptide nucleic acid (PNA) comprises a polypeptide backbone with nucleic acid bases attached as side chains. In embodiments, the PNA comprises a polyamide backbone bearing a plurality of ligands at respective spaced locations along said backbone, said ligands being each independently naturally occurring nucleobases, non-naturally occurring nucleobases or nucleobase-binding groups, each said ligand being bound directly or indirectly to a nitrogen atom in said backbone, and said ligand bearing nitrogen atoms mainly being separated from one another in said backbone by from 4 to 8 intervening atoms.

### The Sugar Group

In embodiments, any nucleotide or abasic within the first nucleotide sequence, the second nucleotide sequence and/or within the connector element independently comprises naturally occurring sugar or a modified sugar.

A modified RNA can include modification of all or some of the sugar groups of the ribonucleic acid. For example, the 2′-hydroxyl group (OH) can be modified or replaced with a number of different “oxy” or “deoxy” substituents. While not being bound by theory, enhanced stability is expected since the hydroxyl can no longer be deprotonated to form a 2′-alkoxide ion. The 2′ alkoxide can catalyze degradation by intramolecular nucleophilic attack on the linker phosphorus atom. While not wishing to be bound by theory, it can be desirable to some embodiments to introduce alterations in which alkoxide formation at the 2′-position is not possible.

Examples of “oxy”-2′-hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′-hydroxyl is connected, e.g., by a methylene bridge or ethylene bridge (e.g., 2′-4′-ethylene bridged nucleic acid (ENA)), to the 4′ carbon of the same ribose sugar; amino, O-AMINE (AMINE=NH₂; alkylamino, dialkylamino, heterocyclyl, arylamino, diaryl amino, heteroaryl amino, diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl, or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substituents are 2′-methoxyethyl, 2′-OCH₃, 2′-O-allyl, 2′-C-allyl, and 2′-fluoro.

The sugar group can also contain one or more carbons that possess the opposite stereochemical configuration than that of the corresponding carbon in ribose. Thus, a modified RNA can include nucleotides containing e.g., arabinose, as the sugar.

Modified RNAs can also include “abasic” sugars, which lack a nucleobase at C-1′. These abasic sugars can also contain modifications at one or more of the constituent sugar atoms.

To maximize nuclease resistance, the 2′ modifications can be used in combination with one or more phosphate linker modifications (e.g., phosphorothioate). The so-called “chimeric” oligonucleotides are those that contain two or more different modifications.

The modification can also entail the wholesale replacement of a ribose structure with another entity (an SRMS) at one or more sites in the nucleic acid agent.

Modified RNA can also include one or more morpholino nucleotides.

### Compounds and Compositions

In embodiments, the first nucleic acid sequence and the second nucleic acid sequence, together with the connector element, is a RIG-I agonist. The RIG-I agonist is capable of inducing interferon production.

In certain embodiments, the RIG-I agonist of the present invention has a double-stranded section of 19 base pairs, 18 base pairs, 17 base pairs, 16 base pairs, 15 base pairs, 14 base pairs, 13 base pairs, 12 base pairs, 11 base pairs, 10 base pairs, or 9 base pairs. In certain embodiments, the double-stranded section comprises about 9, 10, 11, 12, 13, 14, 15, 16, 17, 18, or 19 base pairs. The nucleic acid compound can be of any nucleic acid sequence.

The RIG-I agonist of the invention comprises an element that binds to the first nucleotide sequence and the second nucleotide sequence of the compound.

In certain embodiments, the double-stranded section comprises one or more mispaired bases. That is, Watson-Crick base pairing is not required at each and every nucleotide pair.

In certain embodiments, the RIG-I agonist comprises a nucleotide insertion in in either the first nucleotide sequence or the second nucleotide sequence that remains unpaired in the double-stranded structure and creates a kink. The nucleotide insertion may be a nucleotide insertion of 1-2 nucleotides, preferably of a single nucleotide. In some embodiments, the nucleotide insertion is a nucleotide insertion in the first nucleotide sequence.

Non-limiting examples of nucleic acid compounds of the invention wherein the first and second nucleic acid sequences are connected through a connector element, including exemplary targeting molecules, are as follows:

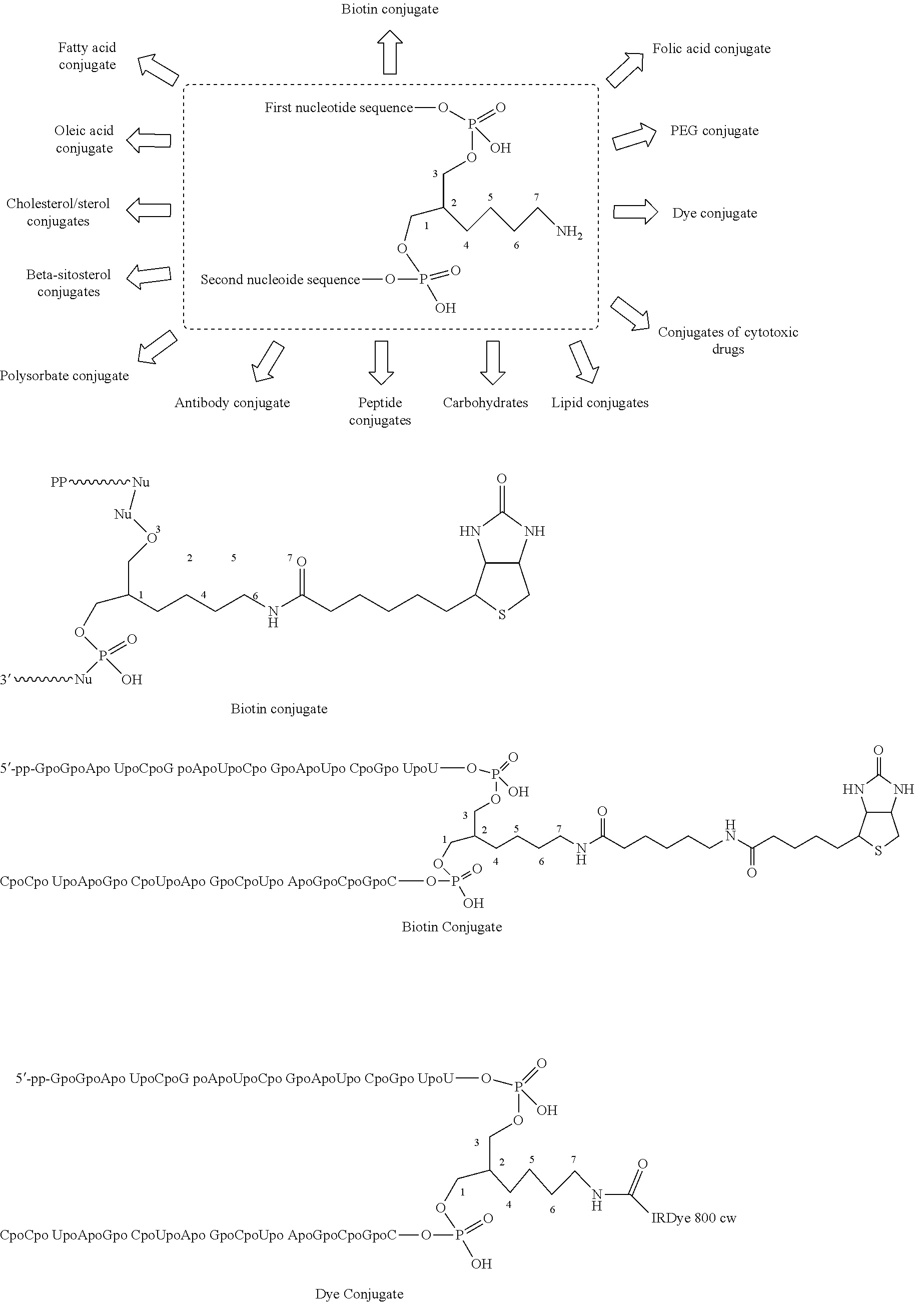

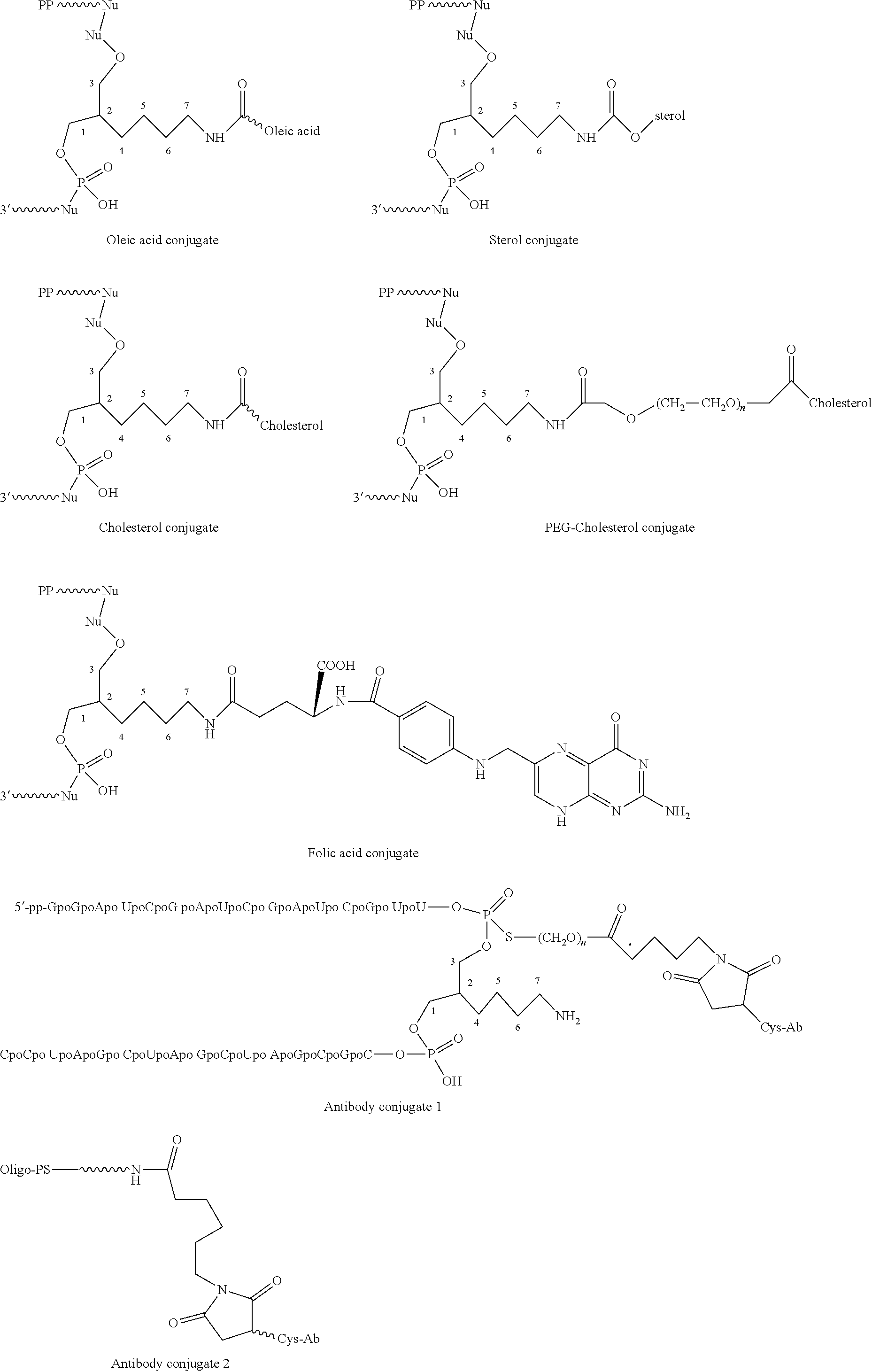

### Pharmaceutical Compositions and Formulations

In another aspect, the invention relates to compositions comprising at least one nucleic acid compound according to the invention and a pharmaceutically acceptable diluent, carrier, solubilizer, emulsifier, preservative and/or adjuvant. Such compositions may comprise one species of such nucleic acid compound or may comprise a plurality of different nucleic acid compounds according to the invention.

The composition can be a pharmaceutical composition, for example an immunostimulatory or antiviral or anti-cancer composition. The immunostimulatory composition may be a vaccine composition further comprising a vaccine, wherein the nucleic acid compound(s) are the adjuvant. If the composition is an antiviral composition, it can further comprise an additional active antiviral agent. If the composition is an anti-cancer composition, it can further comprise an additional active anti-cancer agent.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and/or rate of in vivo clearance of the nucleic acid compound.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising a nucleic acid compound can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising a nucleic acid compound can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a nucleic acid compound, in a pharmaceutically acceptable vehicle. In certain embodiments, a vehicle for parenteral injection is sterile distilled water in which a nucleic acid compound is formulated as a sterile, isotonic solution, and properly preserved. In certain embodiments, the preparation can involve the formulation of the desired molecule with a delivery vehicle or agent, such as injectable microspheres, bio-erodible particles, polymeric compounds (such as polylactic acid, polyglycolic acid or polyethylenimine (e.g., JetPEI®)), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, a nucleic acid compound can be formulated as a dry powder for inhalation. In certain embodiments, an inhalation solution comprising a nucleic acid compound can be formulated with a propellant for aerosol delivery. In certain embodiments, solutions can be nebulized. Pulmonary administration is further described in PCT application No. PCT/US94/001875, which describes pulmonary delivery of chemically modified proteins.

In certain embodiments, it is contemplated that formulations can be administered orally. In certain embodiments, a nucleic acid compound that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of a nucleic acid compound. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of a nucleic acid compound in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

In embodiments, the pharmaceutical composition is an aqueous liquid pharmaceutical formulation suitable for topical administration to the lung or nose comprising (i) a surfactant component which is a mixture of a fatty acid or a pharmaceutically acceptable salt thereof and a non-ionic surfactant and (ii) a polynucleotide molecule according to Formula I or Formula II. In embodiments, the polynucleotide molecule is a polynucleotide molecule according to Formula I. In embodiments, the polynucleotide molecule is a polynucleotide molecule according to Formula II.

By way of definition, as used herein a “fatty acid” refers to a carboxylic acid molecule comprising a carboxylic acid group attached to an aliphatic hydrocarbon “tail”, which is typically between 4 and 24 carbon atoms in length. For example, the aliphatic hydrocarbon “tail” may be between 4 and 22, such as between 4 and 20, such as between 4 and 18, such as between 4 and 16, such as between 4 and 14, such as between 4 and 12, such as between 4 and 10, such as between 4 and 8, such as between 4 and 6, carbon atoms in length. Alternatively, the aliphatic hydrocarbon “tail” may be between 6 and 24, such as between 8 and 24, such as between 10 and 24, such as between 12 and 24, such as between 14 and 24, carbon atoms in length. For example, the aliphatic hydrocarbon “tail” may be between 6 and 22, such as between 6 and 20, such as between 8 and 20, such as between 8 and 18, such as between 10 and 18, carbon atoms in length. In an embodiment, the aliphatic hydrocarbon “tail” is between 4 and 6 carbon atoms in length i.e. the fatty acid is a short-chain fatty acid such as butyric acid (4 carbon atoms). Alternatively, the aliphatic hydrocarbon “tail” is between 6 and 12 carbon atoms in length i.e. the fatty acid is a medium-chain fatty acid such as caprylic acid (8 carbon atoms) and capric acid (10 carbon atoms). Most suitably, the aliphatic hydrocarbon “tail” is between 14 and 24 carbon atoms in length i.e. the fatty acid is a long-chain fatty acid such as oleic acid (18 carbon atoms), stearic acid (18 carbon atoms) and arachidic acid (20 carbon atoms). The aliphatic hydrocarbon “tail” may be saturated or unsaturated. If unsaturated, the aliphatic hydrocarbon “tail” may comprise, for example, one, two, three, four, five, six etc. C═C double bonds, in particular one or two, especially one C═C double bond. Fatty acids may be sub-categorised based on the length and degree of saturation of the aliphatic hydrocarbon “tail”.

Exemplary fatty acids may typically have a molar mass of from about 150 g/mol to about 400 g/mol, for example from about 200 g/mol to about 350 g/mol. They include but are not limited to arachidic acid, arachidonic acid, lauric acid, linoleic acid, linolenic acid, myristic acid, myristoleic acid, oleic acid, palmitic acid, palmitoleic acid, sapienic acid, stearic acid, and vaccenic acid. In particular, the fatty acid is oleic acid.

Suitably the aqueous liquid pharmaceutical formulation comprises a single fatty acid as part of the surfactant component. Alternatively, it comprises a mixture of e.g. of two (or more) fatty acids as part of the surfactant component.

Exemplary non-ionic surfactants may typically have a molar mass of from about 100 g/mol to about 10000 g/mol, in particular from about 100 g/mol to about 2000 g/mol. Exemplary non-ionic surfactants typically comprise one or more polyoxyalkylene moieties e.g. polyoxyethylene and/or polyoxypropylene moieties.

Exemplary non-ionic surfactants include polyoxyalkylenes, particularly poloxamers, such as poloxamer 188, poloxamer 407, poloxamer 171, and poloxamer 185.

Further exemplary non-ionic surfactants include alkyl ethers of polyethylene glycol, such as those known under the brand names Brij 52, Brij 93, Brij 97, Brij L4, Brij 30, and Brij 78.

Additional exemplary non-ionic surfactants include alkylphenyl ethers of polyethylene glycol, such as that known under the brand name Triton X-100.

Particular exemplary non-ionic surfactants include fatty acid esters, such as fatty acid esters of polyols. Such fatty acid esters may comprise one or more e.g. one, two or three fatty acid chains e.g. one fatty acid chain. Specific examples include polyoxyethylene sorbitan fatty acid esters. In particular, the non-ionic surfactant is a polyoxyethylene sorbitan fatty acid ester. Suitable polyoxyethylene sorbitan fatty acid esters include polysorbate 80 (e.g. Tween 80), polysorbate 120, polysorbate 85, polysorbate 65, polysorbate 60, polysorbate 40, and polysorbate 20, in particular polysorbate 80.

Suitably the aqueous liquid pharmaceutical formulation comprises a single non-ionic surfactant as part of the surfactant component. Alternatively, it comprises a mixture of e.g. of two (or more) non-ionic surfactants as part of the surfactant component.

Suitably, the surfactant component is selected from the group consisting of mixtures of (a) oleic acid or a pharmaceutically acceptable salt thereof and a polyoxyethylene sorbitan fatty acid ester, (b) lauric acid or a pharmaceutically acceptable salt thereof and a polyoxyethylene sorbitan fatty acid ester, (c) linoleic acid or a pharmaceutically acceptable salt thereof and a polyoxyethylene sorbitan fatty acid ester, (d) linolenic acid or a pharmaceutically acceptable salt thereof and a polyoxyethylene sorbitan fatty acid ester, (e) palmitic acid or a pharmaceutically acceptable salt thereof and a polyoxyethylene sorbitan fatty acid ester, (f) stearic acid or a pharmaceutically acceptable salt thereof and a polyoxyethylene sorbitan fatty acid ester, (g) oleic acid or a pharmaceutically acceptable salt thereof and a polyoxyalkylene, such as a poloxamer, (h) oleic acid or a pharmaceutically acceptable salt thereof and an alkyl ether of polyethylene glycol, and (i) oleic acid or a pharmaceutically acceptable salt thereof and an alkylphenyl ether of polyethylene glycol.

Most suitably the surfactant component is a mixture of oleic acid or a pharmaceutically acceptable salt thereof and a polyoxyethylene sorbitan fatty acid ester, in particular a polyoxyethylene sorbitan fatty acid ester selected from polysorbate 80 and polysorbate 20. In particular. the surfactant component is a mixture of oleic acid or a pharmaceutically acceptable salt thereof, especially oleic acid, and polysorbate 80.

Pharmaceutically acceptable salt forms of fatty acid that may be employed include sodium, potassium, and ammonium salts, and in particular the sodium salt. Suitably the fatty acid is used as, i.e. is in the form of, the free acid.

The aqueous liquid pharmaceutical formulations of the present invention should suitably form a stable colloidal emulsion. Typically, the stable colloidal emulsion will comprise stable colloidal particles with an average particle size of between about 50 and about 1000 nm, such as between about 50 and about 500 nm, for example between about 50 and about 100 nm, or between about 100 and about 250 nm, or between about 250 and about 500 nm. Thus, for example, the average particle size is between about 100 and 200 nm (see Biophysical Example 1). The aforesaid particle size means hydrodynamic diameter (Z-average size) which can be measured as described in Biophysical Example 1.

Such a formulation is suitably achieved upon use of a surfactant component which is a mixture of a fatty acid, or a pharmaceutically acceptable salt thereof, and a non-ionic surfactant. Such a formulation may more suitably be achieved when the fatty acid is present at a concentration, as provided for in the present invention below, which is at or below (but ideally close to) the critical micellar concentration of said fatty acid. Such a formulation may more suitably be achieved when the non-ionic surfactant is water-miscible when present at a concentration provided for in the present invention below. Physical measurements are suitably made at a temperature of 23 °C and a pressure of 1 standard atmosphere.

Typically, the surfactant component may be present in the formulation at a concentration (meaning the total concentration of the surfactants of the surfactant component) of 0.2-30000 μg/mL, for example 1-30000 μg/mL, for example 1-20000 μg/mL, for example 5-20000 μg/mL, for example 5-15000 μg/mL, for example 5-10000 μg/mL, for example 5-5000 μg/mL. Suitably, the surfactant component is present in the formulation at a concentration of 1-3000 μg/mL, for example 1-2000 μg/mL, for example 5-2000 μg/mL, for example 5-1500 μg/mL, for example 5-1000 μg/mL, for example 5-500 μg/mL. In one embodiment, the surfactant component is present at a concentration of 50-200 μg/mL, for example 75-150 pg/mL, for example 90-120 μg/mL, or about 100 μg/mL. In an alternative embodiment, the surfactant component is present at a concentration of 500-2000 μg/mL, for example 750-1500 μg/mL, for example 900-1200 μg/mL, or about 1000 μg/mL.

Suitably, the fatty acid may be present in the formulation at a concentration of 0.2-30000 μg/mL, for example 1-30000 μg/mL, for example 1-20000 μg/mL, for example 5-10000 μg/mL and the non-ionic surfactant may be present in the formulation at a concentration of 0.2-20000 μg/mL, for example 1-20000 μg/mL, for example 1-15000 μg/mL, for example 5-5000 μg/mL. More suitably, the fatty acid may be present in the formulation at a concentration of 10-100 μg/mL, for example 20-80 μg/mL, for example 25-75 μg/mL, for example 40-60 μg/mL, or about 50 μg/mL, and the non-ionic surfactant may be present in the formulation at a concentration of 10-100 μg/mL, for example 20-80 μg/mL, for example 25-75 μg/mL, for example 30-60 μg/mL, for example 40-50 pg/mL. Alternatively, in another suitable embodiment, the fatty acid may be present in the formulation at a concentration of 100-1000 μg/mL, for example 200-800 μg/mL, for example 250-750 μg/mL, for example 400-600 μg/mL, or about 500 μg/mL, and the non-ionic surfactant may be present in the formulation at a concentration of 100-1000 μg/mL, for example 200-800 μg/mL, for example 250-750 μg/mL, for example 300-600 μg/mL, for example 400-500 μg/mL.

Typically, the surfactant component may be present in the formulation at a concentration of 0.00002% (w/w)-3% (w/w), for example 0.0001% (w/w)-3% (w/w), for example 0.0001% (w/w)-2% (w/w), for example 0.0005% (w/w)-2% (w/w), for example 0.0005% (w/w)-1.5% (w/w), for example 0.0005% (w/w)-1% (w/w), for example 0.0005% (w/w)-0.5% (w/w), wherein the % by weight is with respect to the total weight of the formulation. Suitably, the surfactant component is present in the formulation at a concentration of 0.0001% (w/w)-0.3% (w/w), for example 0.0001% (w/w)-0.2% (w/w), for example 0.0005% (w/w)-0.2% (w/w), for example 0.0005% (w/w)-0.15% (w/w), for example 0.0005% (w/w)-0.1% (w/w), for example 0.0005% (w/w)-0.05% (w/w), wherein the % by weight is with respect to the total weight of the formulation. In one embodiment, the surfactant component is present at a concentration of 0.005% (w/w)-0.02% (w/w), for example 0.0075% (w/w)-0.015% (w/w), for example 0.009% (w/w)-0.012% (w/w), or about 0.01% (w/w), wherein the % by weight is with respect to the total weight of the formulation. In one embodiment, the surfactant component is present at a concentration of 0.05% (w/w)-0.2% (w/w), for example 0.075% (w/w)-0.15% (w/w), for example 0.09% (w/w)-0.12% (w/w), or about 0.1% (w/w), wherein the % by weight Is with respect to the total weight of the formulation.

Suitably, the fatty acid may be present in the formulation at a concentration of 0.00002% (w/w)-3% (w/w), for example 0.0001% (w/w)-3% (w/w), for example 0.0001% (w/w)-2% (w/w), for example 0.0005% (w/w)-1% (w/w), and the non-ionic surfactant may be present in the formulation at a concentration of 0.00002% (w/w)-2% (w/w), for example 0.0001% (w/w)-2% (w/w), for example 0.0001% (w/w)-1.5% (w/w), for example 0.0005% (w/w)-0.5% (w/w), wherein the % by weight is with respect to the total weight of the formulation. More suitably, the fatty acid may be present in the formulation at a concentration of 0.001% (w/w)-0.01% (w/w), for example 0.002% (w/w)-0.008% (w/w), for example 0.0025% (w/w)-0.0075% (w/w), for example 0.004% (w/w)-0.006% (w/w), or about 0.005% (w/w), and the non-ionic surfactant may be present in the formulation at a concentration of 0.001% (w/w)-0.01% (w/w), for example 0.002% (w/w)-0.008% (w/w), for example 0.0025% (w/w)-0.0075% (w/w), for example 0.003% (w/w)-0.006% (w/w), for example 0.004% (w/w)-0.005% (w/w), wherein the % by weight is with respect to the total weight of the formulation. Alternatively, in another suitable embodiment, the fatty acid may be present in the formulation at a concentration of 0.01% (w/w)-0.1% (w/w), for example 0.02% (w/w)-0.08% (w/w), for example 0.025% (w/w)-0.075% (w/w), for example 0.04% (w/w)-0.06% (w/w), or about 0.05% (w/w), and the non-ionic surfactant may be present in the formulation at a concentration of 0.01% (w/w)-0.1% (w/w), for example 0.02% (w/w)-0.08% (w/w), for example 0.025% (w/w)-0.075% (w/w), for example 0.03% (w/w)-0.06% (w/w), for example 0.04% (w/w)-0.05% (w/w), wherein the % by weight is with respect to the total weight of the formulation.

Suitably, the ratio of the amount of fatty acid or a pharmaceutically acceptable salt thereof to non-ionic surfactant, for example wherein each is measured in pg/mL, is between about 5:1 and about 1:5, for example between about 5:1 and about 1:2, for example between about 4:1 and about 1:2, for example between about 2:1 and about 1:2. More suitably, the ratio of the amount of fatty acid or a pharmaceutically acceptable salt thereof to non-ionic surfactant, for example wherein each is measured in pg/mL, is between about 3:2 and about 2:3, for example between about 6:5 and about 1:1, e.g. about 10:9 or about 11:10.

The aqueous liquid pharmaceutical formulations of the invention include water as the solvent. Water includes but not is limited to sterile or purified water, sterile water for injection, RNAse free water, or bacteriostatic water for injection.

Suitably, the aqueous liquid pharmaceutical formulation is substantially free of any solvent or co-solvent other than water. In particular, the aqueous liquid pharmaceutical formulation does not comprise an organic solvent or co-solvent, such as inter alia ethanol, acetone, dimethyl sulfoxide (DMSO), dichloromethane (DCM), N-methyl pyrrolidinone (NMP), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, and benzyl benzoate. As used herein, the term “substantially free of” means that the formulations comprise less than 2% (w/w), for example less than 1% (w/w), such as less than 0.5% (w/w), wherein the % by weight is with respect to the total weight of the formulation. Preferably, the formulation does not comprise any solvent or co-solvent other than water.

The aqueous liquid pharmaceutical formulations according to the present invention may further comprise pharmaceutically acceptable excipients including, but not limited to, antioxidants, buffers, diluents, emulsifiers, lubricants, preservatives, solvents, stabilizers, suspending agents, thickeners, tonicity adjusting (osmotic) agents, vehicles, wetting agents.

Suitable antioxidants include but are not limited to ascorbic acid (vitamin C), glutathione (reduced), lipoic acid, uric acid, carotenes, including β-carotene and retinol (vitamin A), cc-tocopherol (vitamin E), ubiquinol (coenzyme Q), butylated hydroxyanisole, butylated hydroxytoluene, propyl gallate, tert-butylhydroquinone, monothioglycerol, lutein, selenium, manganese, zeaxanthin, or a combination thereof.

The aqueous liquid pharmaceutical formulations of the invention may comprise one or more buffers. Suitable buffers include but are not limited to citrate, borate, formate, glycine, alanine, acetate, aspartate, malate, glyoxylate, gluconate, lactate, glycolate, oxalate, histidine, tartarate and succinate buffer systems. As used herein, references to a “citrate” buffer will be understood to refer to a mixture of citrate and the corresponding acid as a buffer system in a ratio according to the target pH, that is the pH at which the aqueous liquid pharmaceutical formulation is intended to be buffered. For example, the buffer may comprise sodium citrate dihydrate and citric acid monohydrate. In particular, the buffer is based on a weak organic acid, for example the buffer is citrate, acetate, lactate, or formate.

Suitable, pharmaceutically acceptable, diluents include but are not limited to isotonic saline (0.9% w/v), isotonic dextrose (5% w/v), isotonic mixtures of saline and dextrose (e.g. saline (0.45% w/v) and dextrose (2.5% w/v)), sterile or purified water, sterile water for injection or bacteriostatic water for injection. In particular, the diluent is sterile or purified water, sterile water for injection, RNAse free water or bacteriostatic water for injection.

Suitable preservatives include, but are not limited to, edetic acid and alkali salts thereof, such as disodium edetate (also known as “disodium EDTA”) or calcium edetate (also known as calcium EDTA), phenol, m-cresol, chlorocresol, benzyl alcohol, propyl paraben, methyl paraben, butyl paraben, chlorobutanol, phenylethyl alcohol, benzalkonium chloride, thimerosal, propylene glycol, sorbic acid, benzoic acid derivatives and combinations thereof.

Suitable suspending agents include, but are not limited to, acacia (gum), sodium alginate, starch and starch derivatives, xanthan gum, pectin, methylcellulose, hydroxyethylcellulose, sodium carboxymethylcellulose (Avicel RC591), microcrystalline cellulose, hypromellose, hyaluronic acid, and combinations thereof. Particularly suitable suspending agents include, microcrystalline cellulose, sodium carboxymethylcellulose (Avicel RC591), hyaluronic acid, and combinations thereof.

The properties of certain suspending agents may further render them as suitable thickening agents and/or wetting agents. Accordingly, suitable thickening agents and/or wetting agents, may include, but are not limited to the suspending agents recited above. In particular, suitable thickening agents and/or wetting agents include microcrystalline cellulose, sodium carboxymethylcellulose (Avicel RC591), hyaluronic acid, and combinations thereof.

Suitable tonicity adjusting (osmotic) agents include, but are not limited to, polyols, such as sugars and sugar alcohols, for example erythritol, glycerol, lactose, maltitol, mannitol, sorbitol, trehalose, and xylitol, and salts, for example sodium acetate, sodium lactate, sodium chloride, potassium chloride, and calcium chloride. A particularly suitable tonicity adjusting (osmotic) agent is glycerol.

The pH of the aqueous liquid pharmaceutical formulation according to the present invention is suitably between about 4.0 and about 9.0, such as between about 4.0 and about 8.0, such as between about 4.0 and about 7.0 or between about 5.0 and about 8.0. In particular, the pH is suitably between about 4.0 and about 6.0, such as between about 4.0 and about 5.5. For example, the pH of the aqueous liquid pharmaceutical formulation is about 4.0, about 4.1, about 4.2, about 4.3, about 4.4, about 4.5, about, 4.6, about 4.7, about 4.8, about 4.9 or about 5.0. Alternatively, pH is suitably between about 5.5 and about 8.0, such as between about 6.0 and about 8.0, such as between about 6.5 and about 7.5 or between about 7.0 and about 80. For example, the pH of the aqueous liquid pharmaceutical formulation is about 6.5, about 6.6, about 6.7. about 6.8, about 6.9, about 7.0, about 7.1, about 7.2, about 7.3, about 7.4, about 7.5, about 7.6, about 7.7, about 7.8, about 7.9, or about 8.0. The pH of such a pharmaceutical composition may be adjusted by pH adjusting agents including acidifying agents such as hydrochloric acid, tartaric acid, citric acid, succinic acid, phosphoric acid, ascorbic acid, acetic acid, lactic acid, sulphuric acid, formic acid and mixtures thereof, or alkaline buffering agents such as ammonium hydroxide, ethylamine, dipropylamine, triethylamine, alkanediamines, ethanolamines, polyalkylene polyamines, heterocyclic amines, hydroxides of alkali metals, such as sodium and potassium hydroxide, hydroxides of alkali earth metals, such as magnesium and calcium hydroxide, and basic amino acids such as L-arginine, lysine, alanine, leucine, isoleucine, oxylysine and histidine, and mixtures thereof.

It will be understood by the skilled person that an aqueous liquid pharmaceutical formulation suitable for topical administration to the nose may suitably have a pH between about 4.0 and about 9.0. The skilled person would further understand that an aqueous liquid pharmaceutical formulation suitable for topical administration to the lung may suitably have a pH between about 5.5 and about 8.0, such as between about 6.0 and about 8.0, for example between about 7.0 and about 8.0.

Suitably, the aqueous liquid pharmaceutical formulation according to the present invention does not comprise a protein. Furthermore, the aqueous liquid pharmaceutical formulation of the present invention suitably does not comprise a nanoparticle, in particular a lipid nanoparticle (LNP), or a liposome. Moreover, suitably the aqueous liquid pharmaceutical formulation according to the present invention is substantially free of nanoparticle, in particular LNP, and liposome components. For example, suitably, the aqueous liquid pharmaceutical formulation according to the present invention does not comprise a neutral lipid. In particular, the pharmaceutical formulation does not comprise cholesterol, or an analogue thereof. Furthermore, for example, suitably, the aqueous liquid pharmaceutical formulation according to the present invention does not comprise a lipid, other than the fatty acid present therein. In one embodiment in which the fatty acid is oleic acid, suitably the aqueous liquid pharmaceutical formulation does not comprise a lipid, other than oleic acid.

The aqueous liquid pharmaceutical formulations of the present invention are suitable for topical administration the lung or nose. Accordingly, the aqueous liquid pharmaceutical formulations of the present invention are suitable for administration via inhalation, for example suitable for administration topically to the lung via oral inhalation, or for intranasal administration. In one embodiment, the aqueous liquid pharmaceutical formulation of the present invention is administered topically to the lung or nose. Accordingly, in one embodiment, the aqueous liquid pharmaceutical formulations of the present invention are administered via inhalation or are administered intranasally.

It should be noted that the aqueous liquid pharmaceutical formulations of the invention suitable for topical administration to the lung or nose, when administered topically to the lung by oral inhalation or topically to the nose may thereby involve administration to the pharynx.

It will be understood that a formulation suitable for topical administration to the lung may comprise different pharmaceutically acceptable excipients to a formulation suitable for topical administration to the nose. By way of example, a formulation suitable for topical administration to the nose may comprise a suspending and/or wetting and/or thickening agent such as microcrystalline cellulose, sodium carboxymethylcellulose (Avicel RC591), hyaluronic acid, or a combination thereof, whilst a formulation suitable for topical administration to the lung may not.

Suitably, the aqueous liquid pharmaceutical formulations of the present invention are suitable for administration to a mammal. More suitably, the aqueous liquid pharmaceutical formulations of the present invention are suitable for administration to a human. In one embodiment, the aqueous liquid pharmaceutical formulations of the present invention are administered to a mammal. In particular, the aqueous liquid pharmaceutical formulations of the present invention are administered to a human.

Suitably, the aqueous liquid pharmaceutical formulations disclosed herein may be administered to a patient or subject once or more than once a day, for example two times a day, three time a day, four times a day or five times a day. Such treatment may extend for a number of weeks or months.

Additional pharmaceutical compositions will be evident to those skilled in the art, including formulations involving a nucleic acid compound in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, the effective amount of a pharmaceutical composition comprising a nucleic acid compound to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which a nucleic acid compound is being used, the route of administration, and the size (body weight, body surface or organ size) and/or condition (the age and general health) of the patient. In certain embodiments, the clinician can titer the dosage and modify the route of administration to obtain the optimal therapeutic effect.

In certain embodiments, the frequency of dosing will take into account the pharmacokinetic parameters of a nucleic acid compound in the formulation used. In certain embodiments, a clinician will administer the composition until a dosage is reached that achieves the desired effect. In certain embodiments, the composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g., orally, through injection by intravenous, intraperitoneal, intracerebral (intra-parenchymal), intracerebroventricular, intramuscular, subcutaneously, intraocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition comprising a nucleic acid compound in an ex vivo manner. In such instances, cells, tissues and/or organs that have been removed from the patient are exposed to a pharmaceutical composition comprising a nucleic acid compound after which the cells, tissues and/or organs are subsequently implanted back into the patient.

In certain embodiments, a nucleic acid compound can be delivered by implanting certain cells that have been genetically engineered, using methods such as those described herein, to express and secrete the agonist. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

In some aspects, the disclosure provides a pharmaceutical composition comprising a nucleic acid compound according to the invention for stimulating an immune response, treating or delaying progression of a cancer, or reducing or inhibiting tumor growth in a subject in need thereof, and a pharmaceutically acceptable carrier. In some embodiments, the nucleic acid compound is formulated in a polyethylenimine (PEI) carrier. In some embodiments, the PEI carrier is JetPEI®.

In some embodiments, the nucleic acid compounds of the invention comprise a sequence motif in the first nucleotide sequence and/or the second nucleotide, wherein the sequence motif is selected from the group consisting of:
(i) a GT-repeat motif,
(ii) a GA-repeat motif;
(iii) a AUCG-repeat motif;
(iv) an AU-repeat motif,
(v) a dipyrimidine motif;
(vi) a dipurine motif;
(vii) a pyrimidine triplet motif;
(viii) a purine triplet motif;
(ix) a palindromic sequence motif, and
(x) a combination of any of (i)-(ix).

In some embodiments, the nucleic acid compounds of the invention comprise at least one improved biological activity, wherein the improved biological activity is selected from:
(i) an increase in RIG-I-mediated cytokine production;
(ii) an increase in RIG-I-mediated expression of interferon-stimulated genes;
(iii) an increase in RIG-I-mediated intracellular signaling;
(iv) an increase in binding affinity to RIG-Is; and
(v) a combination of any of (i)-(iv).

In some embodiments, the nucleic acid compounds of the invention comprise a sequence motif, wherein the sequence motif is a GT-repeat motif comprises a sequence of <19, about 15-18, about 15, about 10-15, about 10, about 5-10, about 5, about 4 about 18, 17, 16, 15, 14, 13, 12, 11, 10, 9, 8, 7, 6, 5, or 4 guanine and thymine nucleotides, or derivatives or analogs thereof. In some embodiments, the GT-repeat motif is [GT]ₙ, wherein n=2 to 9. In some embodiments, the GT-repeat motif is [GT]₇. In some embodiments, the GT-repeat motif is [GT]₃, and wherein the GT-repeat motif is followed by a purine triplet and UCG, respectively. In some embodiments, the purine triplet is GGA.

In some embodiments, the sequence motif is a GA-repeat motif comprises a sequence of <19, about 15-18, about 15, about 10-15, about 10, about 5-10, about 5, about 4 about 18, 17, 16, 15, 14, 13, 12, 11, 10, 9, 8, 7, 6, 5, or 4 guanine and adenine nucleotides, or derivatives or analogs thereof. In some embodiments, the GA-repeat motif is [GA]ₙ, where n=2 to 9. In some embodiments, the GA-repeat motif is [GA]₇.

In some embodiments, the nucleic acid compounds of the invention comprise a sequence motif, wherein the sequence motif is an AUCG-repeat motif comprising a sequence of <19, about 16, about 12-16, about 12, about 8-12, about 6, about 16, 12, 8 adenine, uracil, cytosine, and guanine nucleotides, or derivatives or analogs thereof.

In some embodiments, the AUCG-repeat motif is [AUCG]ₙ, where n=2 to 4. In some embodiments, the AUCG-repeat motif is [AUCG]₃.

In some embodiments, the AUCG-repeat motif is preceded by a CG or a dipyrimidine motif. In some embodiments, the AUCG-repeat motif is preceded by a CG. In some embodiments, the dipyrimidine motif is CC. In some embodiments, the AUCG-repeat motif is preceded by a dipurine motif. In some embodiments, the dipurine motif is GA. In some embodiments, the dipurine motif is GG.

In some embodiments, the nucleic acid compounds of the invention comprise an AUCG-repeat motif, wherein one or more uridine nucleosides (U) are substituted with a modified nucleoside. In some embodiments, wherein the modified nucleoside is ribothymidine (T). In some embodiments, the AUGC-repeat motif is [AUCG]3, wherein the one or more uridine nucleosides (U) comprising the AUCG-repeat motif are substituted with a modified nucleoside, wherein the modified nucleoside is ribothymidine (T). In some embodiments, the AUGC-repeat motif is [AUCG]3, wherein the one or more uridine nucleosides (U) comprising the AUCG-repeat motif are substituted with a modified nucleoside, wherein the modified nucleoside is ribothymidine (T), and wherein the AUGC-repeat motif is preceded by GG.

In some embodiments, the nucleic acid compounds of the invention comprise an AUCG-repeat motif, wherein one or more guanosine nucleosides (G) are substituted with a modified nucleoside. In some embodiments, the modified nucleoside is inosine (I). In some embodiments, the AUGC-repeat motif is [AUCG]3, wherein the one or more guanosine nucleosides (G) comprising the AUCG-repeat motif are substituted with a modified nucleoside, wherein the modified nucleoside is ribothymidine (T), and wherein the AUGC-repeat motif is preceded by GG.

In some embodiments, the nucleic acid compounds of the invention comprise an AUCG-repeat motif, wherein the motif is preceded by a IG. In some embodiments, the AUCG-repeat motif is [AUCG]3 and is preceded by an IG.

In some embodiments, the nucleic acid compounds of the invention comprise an AUCG-repeat, wherein one or more guanosine nucleosides (G) are substituted with an inosine (I), wherein the AUCG-repeat is preceded by an inosine (I). In some embodiments, the guanosine nucleosides (G) comprising the AUCG-repeat are substituted with an inosine (I), wherein the AUCG-repeat is preceded by an inosine (I), wherein the 5′ most nucleotide of the first polynucleotide comprises inosine (I).

In some embodiments, the 5′ most nucleotide of the first oligonucleotide comprises inosine (I).

In some embodiments, the nucleic acid compounds of the invention comprise an AUCG-repeat sequence motif, wherein the AUCG-repeat motif is [AUCG]2. In some embodiments, the AUCG-repeat motif is preceded by a dipurine motif. In some embodiments, the dipurine motif is GG. In some embodiments, the AUCG-repeat motif is preceded by a purine triplet. In some embodiments, the purine triplet is GGG. In some embodiments, the AUCG-repeat motif is preceded by CCCCCG. In some embodiments, the AUCG-repeat motif is preceded by TCGUCG.

In some embodiments, the nucleic acid compounds of the invention comprise a palindromic sequence, wherein the palindromic sequence comprises a sequence of <19, about 15-18, about 15, about 10-15, about 10, about 18, 17, 16, 15, 14, 13, 12, 11, 10, 9, 8, 7, 6, 5, or 4 nucleotides, or derivatives or analogs thereof, linked in any order that results in a palindrome.

In some embodiments, the linker is flanked by AU. In some embodiments, the linker is flanked by an AU-repeat motif, wherein the AU-repeat motif is [AU]ₙ, where n=2 to 3. In some embodiments, the AU-repeat motif is [AU]2.

In some aspects, the disclosure provides a nucleic acid compound that specifically binds to a RIG-Is, wherein the agonist comprises a blunt-ended, hairpin RNA comprising at least one or more nucleotides comprising inosine which base pairs with cytidine.

In other aspects, the disclosure provides a synthetic RIG-I-like receptor agonist that specifically binds to RIG-I-like receptors, wherein the agonist comprises a blunt-ended, hairpin RNA comprising a non-nucleotide linker.

In some embodiments, inosine, if present, base pairs with cytidine.

In some aspects, the nucleic acid compounds of the invention exhibits at least one or more of the following properties:
(a) specifically binds to RIG-I;
(b) increases RIG-I-mediated cytokine production;
(c) increases RIG-I-mediated expression of interferon-stimulated genes (ISGs);
(d) increases RIG-I-dependent intracellular signaling;
(e) increases stability of the duplex;
(f) increases binding affinity to RIG-Is;
(g) decreases off-target binding;
(h) increases biological half-life;
(i) increases biodistribution and bioavailability;
(j) increases and/or enhances uptake into cells and/or tissues;
(k) decreases immunogenicity; and
(l) a combination of any of (a)-(k).

In some aspects, the nucleic acid compound of the invention is a synthetic RIG-I-like receptor (RLR) agonist that specifically binds to a RIG-I-like receptor (RLR), wherein the agonist comprises a blunt-ended, and wherein the nucleic acid compound comprises at least one inosine nucleoside, and wherein the inosine nucleoside base pairs with cytidine in the hairpin RNA.

In some embodiments, the nucleic acid compounds of the invention comprises a modified nucleotide, a modified nucleoside, or a modified nucleobase, or a combination thereof. In some embodiments, the agonist comprises a modification to the internucleotide linkages or to the polynucleotide backbone.

### Methods of Making Nucleic Acid Compounds of the Invention

Nucleic acid compounds of the invention may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, nucleic acid compounds are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe a nucleic acid compound described herein.

Non-natural modified nucleobases may be introduced into polynucleotides, e.g., RNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990). The synthesis of oligonucleotides, polynucleotides, and conjugations and ligations thereof, is further described in Taskova et al., (2017) Chembiochem 18(17):1671-1682; Gooding et al., (2016) Eur J Pharm Biopharm 107:321-40; Menzi et al., (2015) Future Med Chem 7(13):1733-49; Winkler J., (2013) Ther Deliv. (7):791-809; Singh et al., (2010) Chem Soc Rev 39(6):2054-70; and Lu et al., (2010) Bioconjug Chem 21(2):187-202.

### Applications

The compositions described herein can be used in diagnostic and therapeutic applications. For example, detectably-labeled nucleic acid compounds can be used in assays to detect the presence or amount of the target protein in a sample (e.g., a biological sample). The compositions can be used in in vitro assays for studying inhibition of target function (e.g., RIG-I-mediated cellular signaling or response). In some embodiments, e.g., in which the compositions bind to and activate a target (e.g., a protein or polypeptide), the compositions can be used as positive controls in assays designed to identify additional novel compounds that also induce activity of the target protein or polypeptide and/or are otherwise are useful for treating a disorder associated with the target protein or polypeptide. For example, a RIG-I-activating composition can be used as a positive control in an assay to identify additional compounds (e.g., small molecules, aptamers, or antibodies) that induce, increase, or stimulate RIG-I function. The compositions can also be used in therapeutic methods as elaborated on below.

The invention further encompasses the use of the nucleic acid compound(s) of the invention as an adjuvant or as an antiviral agent or as an anti-cancer agent. Also contemplated is the nucleic acid compound of the invention or composition of the invention for use in a method for stimulating the immune system or treating/preventing a viral infection or treating or preventing cancer in a subject in need thereof. The nucleic acid compound(s) can act as adjuvants for the active agent or can be used for their own antiviral or anti-cancer activity. In some aspects, the nucleic acid compound can be added as an adjuvant formulated either together or added separately to a vaccine for infections or cancer vaccine or other vaccines used to stimulate the immune system.

Another aspect of the invention features a method for stimulating the immune system in a subject in need thereof, the method comprising administering an effective amount of the nucleic acid compound according to the invention or of the composition of the invention to said subject. Another method of the invention is for treating or preventing a viral infection in a subject in need thereof, the method comprising administering an effective amount of the nucleic acid compound according to the invention or of the composition of the invention to said subject. A still further method of the invention is for treating or preventing cancer in a subject in need thereof, the method comprising administering an effective amount of the nucleic acid compound according to the invention or of the composition of the invention to said subject.

### Methods of Use

The compositions of the present invention have numerous in vitro and in vivo utilities involving the detection and/or quantification of RIG-Is and/or the agonism of RIG-I function.

The above-described compositions are useful in, inter alia, methods for treating or preventing a variety of cancers or infectious diseases in a subject. The compositions can be administered to a subject, e.g., a human subject, using a variety of methods that depend, in part, on the route of administration. The route can be, e.g., intravenous injection or infusion (IV), subcutaneous injection (SC), intradermal injection (ID), intraperitoneal (IP) injection, intramuscular injection (IM), intratumoral injection (IT) or intrathecal injection. The injection can be in a bolus or a continuous infusion.

The nucleic acid compound of the invention are capable of inducing interferon, for example, type I interferon, production in a cell.

Accordingly, the present invention provides the use of the nucleic acid compound of the invention for preventing and/or treating diseases or conditions in which inducing IFN production would be beneficial, such as infections, tumors/cancers, inflammatory diseases, and disorders, and immune disorders.

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some embodiments, a nucleic acid compound is therapeutically delivered to a subject by way of local administration.

A suitable dose of a nucleic acid compound described herein, which dose is capable of treating or preventing cancer in a subject, can depend on a variety of factors including, e.g., the age, sex, and weight of a subject to be treated and the particular inhibitor compound used. Other factors affecting the dose administered to the subject include, e.g., the type or severity of the cancer or infectious disease. For example, a subject having metastatic melanoma may require administration of a different dosage of a nucleic acid compound than a subject with glioblastoma. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will also depend upon the judgment of the treating medical practitioner (e.g., doctor or nurse). Suitable dosages are described herein.

A pharmaceutical composition can include a therapeutically effective amount of a nucleic acid compound thereof described herein. Such effective amounts can be readily determined by one of ordinary skill in the art based, in part, on the effect of the administered nucleic acid compound, or the combinatorial effect of the nucleic acid compound and one or more additional active agents, if more than one agent is used. A therapeutically effective amount of a nucleic acid compound described herein can also vary according to factors such as the disease state, age, sex, and weight of the individual, and the ability of the agonist (and one or more additional active agents) to elicit a desired response in the individual, e.g., reduction in tumor growth. For example, a therapeutically effective amount of a nucleic acid compound can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the nucleic acid compounds described herein can further be evaluated in, e.g., Phase I dose escalation studies. See, e.g., van Gurp et al. (2008) Am J Transplantation 8 (8): 1711-1718; Hanouska et al. (2007) Clin Cancer Res 13(2, part 1):523-531; and Hetherington et al. (2006) Antimicrobial Agents and Chemotherapy 50(10): 3499-3500.

In some embodiments, the composition contains any of the nucleic acid compounds described herein and one or more (e.g., two, three, four, five, six, seven, eight, nine, 10, or 11 or more) additional therapeutic agents such that the composition as a whole is therapeutically effective. For example, a composition can contain a nucleic acid compound described herein and an alkylating agent, wherein the agonist and agent are each at a concentration that when combined are therapeutically effective for treating or preventing a cancer (e.g., melanoma) in a subject.

Toxicity and therapeutic efficacy of such compositions can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A nucleic acid compound that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. For a nucleic acid compound described herein, the therapeutically effective dose can be estimated initially from cell culture assays. A dose can be formulated in animal models to achieve a circulating plasma concentration range that includes the EC₅₀ (i.e., the concentration of the agonist which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

In some embodiments, the methods can be performed in conjunction with other therapies for cancer or infectious disease. For example, the composition can be administered to a subject at the same time, prior to, or after, radiation, surgery, targeted or cytotoxic chemotherapy, chemoradiotherapy, hormone therapy, immunotherapy, gene therapy, cell transplant therapy, precision medicine, genome editing therapy, or other pharmacotherapy.

As described above, the compositions described herein (e.g., nucleic acid compound compositions) can be used to treat a variety of cancers such as but not limited to: Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma, marginal zone B cell lymphoma, polycythemia vera, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, rectal cancer; cancer of the respiratory system, sarcoma, skin cancer, stomach cancer, testicular cancer, thyroid cancer, uterine cancer, and cancer of the urinary system.

In some aspects, the disclosure provides a method to increase RIG-I-mediated production of one or more cytokines in a cell, the method comprising contacting the cell with a nucleic acid compound provided by the invention, wherein the agonist increases RIG-I-mediated cytokine production in a cell.

In some aspects, the disclosure provides a method to increase RIG-I-mediated expression of one or more interferon-stimulated genes in a cell, the method comprising contacting the cell with a nucleic acid compound provided by the invention, wherein the agonist increases RIG-I-mediated expression of one or more interferon-stimulated genes in a cell.

In some aspects, the disclosure provides a method to increase RIG-I-dependent intracellular signaling in a cell, the method comprising contacting the cell with a nucleic acid compound provided by the invention, wherein the agonist increases RIG-I-dependent intracellular signaling.

In some aspects, the disclosure provides a method of stimulating an immune response in a subject, the method comprising administering to the subject an effective amount of a nucleic acid compound provided by the invention, or a pharmaceutical composition provided by the invention.

In some aspects, the disclosure provides a method of treating or delaying progression of a cancer in a subject, the method comprising administering to the subject an effective amount of a nucleic acid compound provided by the invention, or a pharmaceutical composition provided by the invention.

In some aspects, the disclosure provides a method of reducing or inhibiting tumor growth in a subject in need thereof, the method comprising administering to the subject an effective amount of a nucleic acid compound provided by the invention, or a pharmaceutical composition provided by the invention.

In some aspects, the disclosure provides a method for stimulating an immune response, treating or delaying progression of a cancer, or inhibiting tumor growth in a subject in need thereof, the method comprising administering to the subject an effective amount of a nucleic acid compound provided by the invention, or a pharmaceutical composition provided by the invention, wherein the compound, or the pharmaceutical composition increases RIG-I-mediated production of one or more cytokines in a cell, increases RIG-I-mediated expression of one or more interferon-stimulated genes in a cell, and or increases RIG-I-dependent intracellular signaling in a cell, thereby stimulating the immune response, treating or delaying progression of the cancer, or inhibiting growth of the tumor.

In some aspects, the disclosure provides a method for treating, ameliorating, and/or preventing viral infections caused by RNA or DNA viruses, and/or ameliorating, minimizing, reversing, and/or preventing persistent viral infection, and/or minimize or prevent viral infection-derived mortality and/or lethality, in a subject, the method comprising administering to the subject a therapeutically effective amount of a nucleic acid compound of the invention. In embodiments, the subject is a tumor-bearing subject. In embodiments, the administering induces type I interferon production in at least one cell of the subject.

In embodiments, the administering takes place before the subject is exposed to the virus. In embodiments, administering takes place after the subject is exposed to the virus. In embodiments, the administering reduces, minimizes, and/or prevents viral replication in the subject. In embodiments, the virus can include positive and negative stranded RNA viruses or DNA viruses.

In any of the methods described herein, the administering reduces recovery time for, eliminates, or minimizes at least one complication from the viral infection.

In any of the methods described herein, the at least one complication comprises at least one of weight loss, fever, cough, fatigue, muscle and/or body ache, nausea, vomiting, diarrhea, shortness of breath, loss of smell and/or taste, acute respiratory distress syndrome (ARDS), low blood oxygen levels, pneumonia, multi-organ failure, septic shock, heart failure, arrhythmias, heart inflammation, blood clots, and death.

In embodiments, the virus comprises at least one of hepatitis C virus, hepatitis B virus, influenza virus, herpes simplex virus (HSV), human immunodeficiency virus (HIV), respiratory syncytial virus (RSV), vesicular stomatitis virus (VSV), cytomegalovirus (CMV), poliovirus, encephalomyocarditis virus (EMCV), human papillomavirus (HPV), and smallpox virus.

In embodiments, the virus comprises an Orthomyxoviridae virus. In embodiments, the Orthomyxoviridae virus comprises at least one of an Alphainfluenzavirus, Betainfluenzavirus, Deltainfluenzavirus, Gammainfluenzavirus, Isavirus, Thogotovirus, and Quaranjavirus. In embodiments, the Alphainfluenzavirus comprises at least one of Influenza A virus, Influenza B virus, and Influenza C virus.

In embodiments, the virus comprises a Coronavirus. In embodiments, the Coronavirus comprises at least one of MERS-CoV, SARS-CoV, and SARS-CoV 2.

In embodiments, the SARS-CoV-2 infection is caused by at least one variant strain of SARS-CoV-2. In embodiments, the SARS-CoV-2 comprises at least one variant selected from B.1.1.7 (Alpha), B.1.351 (Beta), P.1 (Gamma), B.1.617.2 (Delta), B.1.429/B.1.427 (Epsilon), B.1.617.1 (Kappa), B.1.525 (Eta), B.1.526 (Iota), P.3 (Theta), P.2 (Zeta), and B.1.1.529 (Omicron).

In embodiments, the SARS-CoV-2 comprises at least one variant selected from A.1-A.6, B.3-B.7, B.9, B.10, B.13-B.16, B.2, B.1 lineage, P.1, P.2, P.3, and R.1.

In embodiments, the B.1 lineage comprises at least one of (including, but not limited to, B.1, B.1.1, B.1.1.7, B.1.1.7 with E484K, B.1.2, B.1.5-B.1.72, B.1.9, B.1.13, B.1.22, B.1.26, B.1.37, B.1.3-B.1.66, B.1.177, B.1.243, B.1.313, B.1.351, B.1.427, B.1.429, B.1.525, B.1.526, B.1.526.1, B.1.526.2, B.1.617, B.1.617.1, B.1.617.2, B.1.617.3, B.1.619, B.1.620, and B.1.621.

In any of the methods described herein, wherein the subject suffers from long COVID.

As described herein, the nucleic acid compound is useful for treating a tumor, wherein the tumor comprises a cancer selected from biliary tract cancer, brain cancer, breast cancer, cervical cancer, choriocarcinoma, colon cancer, endometrial cancer, esophageal cancer, gastric cancer, intraepithelial neoplasm, leukemia, lymphoma, liver cancer, lung cancer, melanoma, myelomas, neuroblastoma, oral cancer, ovarian cancer, pancreatic cancer, prostate cancer, rectal cancer, sarcoma, skin cancer, testicular cancer, thyroid cancer, or renal cancer.

In embodiments, the tumor comprises a cancer selected from hairy cell leukemia, chronic myelogenous leukemia, cutaneous T-cell leukemia, chronic myeloid leukemia, non-Hodgkin's lymphoma, multiple myeloma, follicular lymphoma, malignant melanoma, squamous cell carcinoma, renal cell carcinoma, prostate carcinoma, bladder cell carcinoma, breast carcinoma, ovarian carcinoma, non-small cell lung cancer, small cell lung cancer, hepatocellular carcinoma, basalioma, colon carcinoma, cervical dysplasia, and Kaposi's sarcoma (AIDS-related and non-AIDS related).

Alternatively, a polynucleotide molecule according to Formula II, in particular an antisense oligonucleotide or a siRNA molecule, may be intended to down-regulate, reduce, silence or knock-down expression of an endogenous gene, in particular when said gene, and consequently the encoded protein (or other gene product), is over-expressed and said over-expression is contributing to cellular dysfunction, or when said gene, and consequently the encoded protein (or other gene product) is defective or dysfunctional and therefore contributing to cellular dysfunction.

Therefore, in one embodiment there is provided an aqueous liquid pharmaceutical formulation for use according to the present invention, wherein the aqueous liquid pharmaceutical formulation decreases the endogenous expression of a protein (or other gene product). As used herein, the term “decreases” includes restoring i.e. decreasing from a high value to a “normal” value and impairing i.e., decreasing from a “normal” value to a low value or zero e.g. silencing, endogenous gene expression.

Accordingly, in one embodiment, an aqueous liquid pharmaceutical formulation for use according to the present invention, wherein the polynucleotide molecule is a polynucleotide molecule which decreases the endogenous expression of a protein (or other gene product), is for use in the treatment of a disease or condition which is treated by decreasing the endogenous expression of a protein (or other gene product).

In an alternative embodiment, the present invention provides a method for the treatment of a disease or condition, which is treated by decreasing the endogenous expression of a protein (or other gene product), comprising administering to a subject in need thereof a therapeutically or prophylactically effective amount of an aqueous liquid pharmaceutical formulation as described herein, wherein the polynucleotide molecule is a polynucleotide molecule which decreases the endogenous expression of a protein (or other gene product).

Moreover, the present invention provides use of an aqueous liquid pharmaceutical formulation as described herein, wherein the polynucleotide molecule is a polynucleotide molecule which decreases the endogenous expression of a protein (or other gene product), in the manufacture of a medicament for use in the treatment of a disease or condition which is treated by decreasing the endogenous expression of a protein (or other gene product).

In one embodiment, a disease or condition treated by decreasing the endogenous expression of a protein (or other gene product) is an infectious disease. The infectious disease is suitably bacterial, fungal, parasitic, or viral in origin. In particular, the infectious disease is infection by a virus or disease associated with infection with such a virus. Suitably, virus infects the respiratory tract and the disease associated with viral infection is a disease of the respiratory tract.

In embodiments, the nucleic acid compound according to the invention or a composition thereof is suitable for use as medicament.

In embodiments, the nucleic acid compound according to the invention or a composition thereof is used in the treatment of a disease or condition which is treated by stimulation or activation of the innate and/or adaptive immune system and/or the raising of an innate and/or adaptive immune response.

In embodiments, the invention provides a method for the treatment of a disease or condition which is treated by stimulation or activation of the innate and/or adaptive immune system and/or the raising of an innate and/or adaptive immune response, comprising administering to a subject in need thereof a therapeutically or prophylactically effective amount of one or more nucleic acid compounds according to the invention or a composition thereof.

In embodiments of any of the uses or methods described herein, the disease or condition is infection by a virus or associated with infection with such a virus. In embodiments, the virus infects the respiratory tract and the disease associated with infection is a disease of the respiratory tract.

In embodiments of any of the uses or methods described herein, the disease or condition is cancer.

In any of the methods described herein, the subject can be an immune-compromised and/or immunodeficient subject.

In any of the methods, the nucleic acid compound of the invention can be combined with antiviral agents: such as protease inhibitors, polymerase inhibitors, integrase inhibitors, viral entry blocking agents, antiviral antibodies and so on.

Combinations of Nucleic Acid Compounds with Additional Therapeutic Agents

In some embodiments, a nucleic acid compound described herein can be administered to a subject as a monotherapy. Alternatively, the nucleic acid compound can be administered to a subject as a combination therapy with another treatment, e.g., another treatment for a cancer. For example, the combination therapy can include administering to the subject (e.g., a human patient) one or more additional agents that provide a therapeutic benefit to a subject who has, or is at risk of developing, cancer.

In some embodiments of the methods provided by the disclosure, the nucleic acid compound or pharmaceutical composition is administered in combination with one or more additional therapeutic agents, wherein the one or more additional therapeutic agents is selected from the group consisting of: a chemotherapy, a targeted anti-cancer therapy, an oncolytic drug, a cell death-inducing agent, an opsonizing agent (e.g., an opsonizing antibody) a cytotoxic agent, an immune-based therapy, a cytokine, an activator or agonist of a costimulatory molecule, an inhibitor of an inhibitory molecule, a vaccine, a cellular immunotherapy, or a combination thereof.

In some embodiments, the nucleic acid compound or pharmaceutical composition is administered preceding or subsequent to administration of the one or more additional therapeutic agents or wherein the one or more additional therapeutic agents is administered concurrently with, preceding or subsequent to the administration of the agonist or pharmaceutical composition.

In some embodiments, the one or more additional therapeutic agents is a immune checkpoint inhibitor. In some embodiments, the one or more additional therapeutic agents is a PD-1/PD-L1 antagonist, a TIM-3 antagonist, a VISTA antagonist, an adenosine A2AR antagonist, a B7-H3 antagonist, a B7-H4 antagonist, a BTLA antagonist, a CTLA-4 antagonist, an IDO antagonist, a KIR antagonist, a LAG-3 antagonist, a toll-like receptor 3 (TLR3) agonist, a toll-like receptor 7 (TLR7) agonist, a toll-like receptor 9 (TLR9) agonist.

Combination with Chemotherapeutic Agents

Chemotherapeutic agents suitable for combination and/or co-administration with compositions of the present invention include, for example: taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g. mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II) (DDP), procarbazine, altretamine, cisplatin, carboplatin, oxaliplatin, nedaplatin, satraplatin, or triplatin tetranitrate), anthracycline (e.g. daunorubicin (formerly daunomycin) and doxorubicin), antibiotics (e.g. dactinomcin (formerly actinomycin), bleomycin, mithramycin, and anthramycin (AMC)), and anti-mitotic agents (e.g. vincristine and vinblastine) and temozolomide.

Combination with PD-1/PD-L1 Antagonists

In some embodiments, a nucleic acid compound, or pharmaceutical compositions thereof, provided by the disclosure is combined (e.g., administered in combination) with one or more PD-1/PD-L1 antagonist that specifically binds to human PD-1 or PD-L1 and inhibits PD-1/PD-L1 biological activity and/or downstream pathway(s) and/or cellular processed mediated by human PD-1/PD-L1 signaling or other human PD-1/PD-L1-mediated functions.

Accordingly, provided herein are PD-1/PD-L1 antagonists that directly or allosterically block, antagonize, suppress, inhibit or reduce PD-1/PD-L1 biological activity, including downstream pathways and/or cellular processes mediated by PD-1/PD-L1 signaling, such as receptor binding and/or elicitation of a cellular response to PD-1/PD-L1. Also provided herein are PD-1/PD-L1 antagonists that reduce the quantity or amount of human PD-1 or PD-L1 produced by a cell or subject.

In some embodiments, the disclosure provides a PD-1/PD-L1 antagonist that binds human PD-1 and prevents, inhibits or reduces PD-L1 binding to PD-1. In some aspects, the PD-1/PD-L1 antagonist binds to the mRNA encoding PD-1 or PD-L1 and prevents translation. In some embodiments, the PD-1/PD-L1 antagonist binds to the mRNA encoding PD-1 or PD-L1 and causes degradation and/or turnover.

In some embodiments, the PD-1/PD-L1 antagonist inhibits PD-1 signaling or function. In some embodiments, the PD-1/PD-L1 antagonist blocks binding of PD-1 to PD-L1, PD-L2, or to both PD-L1 and PD-L2. In some embodiments, the PD-1/PD-L1 antagonist blocks binding of PD-1 to PD-L1. In some embodiments, the PD-1/PD-L1 antagonist blocks binding of PD-1 to PD-L2. In some embodiments, the PD-1/PD-L1 antagonist blocks the binding of PD-1 to PD-L1 and PD-L2. In some embodiments, the PD-1/PD-L1 antagonist specifically binds PD-1. In some embodiments, the PD-1/PD-L1 antagonist specifically binds PD-L1. In some embodiments, the PD-1/PD-L1 antagonist specifically binds PD-L2.

In some embodiments, the PD-1/PD-L1 antagonist inhibits the binding of PD-1 to its cognate ligand. In some embodiments, the PD-1/PD-L1 antagonist inhibits the binding of PD-1 to PD-L1, PD-1 to PD-L2, or PD-1 to both PD-L1 and PD-L2. In some embodiments, the PD-1/PD-L1 antagonist does not inhibit the binding of PD-1 to its cognate ligand.

In some embodiments, the PD-1/PD-L1 antagonist is an isolated monoclonal antibody (mAb), or antigen binding fragment thereof, which specifically binds to PD-1 or PD-L1. In some embodiments, the PD-1/PD-L1 antagonist is an antibody or antigen binding fragment thereof that specifically binds to human PD-1. In some embodiments, the PD-1/PD-L1 antagonist is an antibody or antigen binding fragment thereof that specifically binds to human PD-L1. In some embodiments, the PD-1/PD-L1 antagonist is an antibody or antigen binding fragment that binds to human PD-L1 and inhibits the binding of PD-L1 to PD-1. In some embodiments, the PD-1/PD-L1 antagonist is an antibody or antigen binding fragment that binds to human PD-1 and inhibits the binding of PD-L1 to PD-1.

Several immune checkpoint antagonists that inhibit or disrupt the interaction between PD-1 and either one or both of its ligands PD-L1 and PD-L2 are in clinical development or are currently available to clinicians for treating cancer.

Examples of anti-human PD-1 monoclonal antibodies, or antigen binding fragments thereof, that may comprise the PD-1/PD-L1 antagonist in any of the compositions, methods, and uses provided by the disclosure include, but are not limited to: KEYTRUDA® (pembrolizumab, MK-3475, h409A11; see U.S. Pat. Nos. 8,952,136, 8,354,509, 8,900,587, and EP2170959, all of which are included herein by reference in their entirety; Merck), OPDIVO® (nivolumab, BMS-936558, MDX-1106, ONO-4538; see U.S. Pat. Nos. 7,595,048, 8,728,474, 9,073,994, 9,067,999, EP1537878, U.S. Pat. Nos. 8,008,449, 8,779,105, and EP2161336, all of which are included herein by reference in their entirety; Bristol Myers Squibb), MEDI0680 (AMP-514), BGB-A317 and BGB-108 (BeiGene), 244C₈ and 388D4 (see WO2016106159, which is incorporated herein by reference in its entirety; Enumeral Biomedical), PDR001 (Novartis), and REGN2810 (Regeneron). Accordingly, in some embodiments the PD-1/PD-L1 antagonist is pembrolizumab. In some embodiments, the PD-1/PD-L1 antagonist is nivolumab.

Examples of anti-human PD-L1 monoclonal antibodies, or antigen binding fragments thereof, that may comprise the PD-1/PD-L1 antagonist in any of the compositions, methods, and uses provided by the disclosure include, but are not limited to: BAVENCIO® (avelumab, MSB0010718C, see WO2013/79174, which is incorporated herein by reference in its entirety; Merck/Pfizer), IMFINZI® (durvalumab, MEDI4736), TECENTRIQ® (atezolizumab, MPDL3280A, RG7446; see WO2010/077634, which is incorporated herein by reference in its entirety; Roche), MDX-1105 (BMS-936559, 12A4; see U.S. Pat. No. 7,943,743 and WO2013/173223, both of which are incorporated herein by reference in their entirety; Medarex/BMS), and FAZ053 (Novartis). Accordingly, in some embodiments the PD-1/PD-L1 antagonist is avelumab. In some embodiments, the PD-1/PD-L1 antagonist is durvalumab. In some embodiments, the PD-1/PD-L1 antagonist is atezolizumab.

In some embodiments, the PD-1/PD-L1 antagonist is an immunoadhesin that specifically bind to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010/027827 and WO2011/066342, both of which are incorporated herein by reference in their entirety. In some embodiments, the PD-1/PD-L1 antagonist is AMP-224 (also known as B7-DCIg), which is a PD-L2-FC fusion protein that specifically binds to human PD-1.

It will be understood by one of ordinary skill that any PD-1/PD-L1 antagonist which binds to PD-1 or PD-L1 and disrupts the PD-1/PD-L1 signaling pathway, is suitable for compositions, methods, and uses disclosed herein.

In some embodiments, the PD-1/PD-L1 antagonist is a small molecule, a nucleic acid, a peptide, a peptide mimetic, a protein, a carbohydrate, a carbohydrate derivative, or a glycopolymer. Exemplary small molecule PD-1 inhibitors are described in Zhan et al., (2016) Drug Discov Today 21(6):1027-1036.

In some embodiments of the methods provided by the disclosure, the nucleic acid compound is combined with a PD-1/PD-L1 antagonist, wherein the PD-1/PD-L1 antagonist is selected from the group consisting of: PDR001, KEYTRUDA® (pembrolizumab), OPDIVO® (nivolumab), pidilizumab, MEDIO680, REGN2810, TSR-042, PF-06801591, and AMP-224. In some embodiments, the PD-1/PD-L1 antagonist is selected from the group consisting of: FAZ053, TENCENTRIQ® (atezolizumab), BAVENCIO® (avelumab), IIFINZI® (durvalumab), and BMS-936559.

Combinations with TIM-3 Antagonist

In some embodiments, a nucleic acid compound, or pharmaceutical compositions thereof, provided by the disclosure is combined (e.g., administered in combination) with a TIM-3 antagonist. The TIM-3 antagonist may be an antibody, an antigen binding fragment thereof, an immunoadhesin, a fusion protein, or an oligopeptide. In some embodiments, the TIM-3 antagonist is chosen from MGB453 (Novartis), TSR-022 (Tesaro), or LY3321367 (Eli Lilly).

Combinations with LAG-3 Antagonist

In some embodiments, a nucleic acid compound, or pharmaceutical compositions thereof, provided by the disclosure is combined (e.g., administered in combination) with a LAG-3 antagonist. The LAG-3 antagonist may be an antibody, an antigen binding fragment thereof, an immunoadhesin, a fusion protein, or oligopeptide. In some embodiments, the LAG-3 inhibitor is chosen from LAG525 (Novartis), BMS-986016 (Bristol-Myers Squibb), TSR-033 (Tesaro), MK-4280 (Merck & Co), or REGN3767 (Regeneron).

Combinations with Toll-Like Receptor (TLR) Agonists

In some embodiments, a nucleic acid compound, or pharmaceutical composition thereof, provided by the disclosure is combined (e.g., administered in combination) with a TLR agonist.

Toll-like receptors (TLRs) are a family of germline-encoded transmembrane proteins that facilitate pathogen recognition and activation of the innate immune system. (Hoffmann et al., (1999) Science 284:1313-1318; Rock et al., (1998) Proc Natl Acad Sci USA 95:588-593). TLRs are pattern recognition receptors (PRRs), and are expressed by cells of the innate immune system. Examples of known ligands for TLRs include gram positive bacteria (TLR-2), bacterial endotoxin (TLR-4), flagellin protein (TLR-5), bacterial DNA (TLR-9), double-stranded RNA and poly I:C (TLR-3), and yeast (TLR-2). In vivo activation of TLRs initiates an innate immune response involving specific cytokines, chemokines and growth factors. While all TLRs can activate certain intracellular signaling molecules such as nuclear factor kappa beta (NF-κB) and mitogen activated protein kinases (MAP kinases), the specific set of cytokines and chemokines released appears to be unique for each TLR. TLR7, 8, and 9 comprise a subfamily of TLRs which are located in endosomal or lysosomal compartments of immune cells such as dendritic cells and monocytes. In contrast to TLR7 and 9 which are highly expressed on plasmacytoid dendritic cells (pDC), TLR8 is mainly expressed on myeloid DC (mDC) and monocytes. This subfamily mediates recognition of microbial nucleic acids, such as single stranded RNA.

Small, low-molecular weight (less than 400 Daltons) synthetic imidazoquinoline compounds which resemble the purine nucleotides adenosine and guanosine were the first TLR7 and TLR8 agonists to be identified. A number of these compounds have demonstrated anti-viral and anti-cancer properties. For example, the TLR7 agonist imiquimod (ALDARA™) was approved by the U.S. Food and Drug Administration as a topical agent for the treatment of skin lesions caused by certain strains of the human papillomavirus. Imiquimod may also be useful for the treatment of primary skin cancers and cutaneous tumors such as basal cell carcinomas, keratoacanthomas, actinic keratoses, and Bowen's disease. The TLR7/8 agonist resiquimod (R-848) is being evaluated as a topical agent for the treatment of human genital herpes.

TLR agonists according to the disclosure can be any TLR agonist. For example, a TLR agonist can encompass a natural or synthetic TLR ligand, a mutein or derivative of a TLR ligand, a peptide mimetic of a TLR ligand, a small molecule that mimics the biological function of a TLR ligand, or an antibody that stimulates a TLR receptor. A TLR ligand is any molecule that binds to a TLR.

In some embodiments, a nucleic acid compound, or pharmaceutical composition thereof, provided by the disclosure, is combined with a TLR agonist, wherein the TLR agonist is selected from the group consisting of: a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, and a TLR11 agonist.

In some embodiments, a nucleic acid compound provided by the invention is combined with a TLR3 agonist. A TLR3 agonist is an agonist that causes a signaling response through TLR3. Exemplary TLR3 agonists include, but are not limited to, polyinosinic:polycytidylic acid (poly I:C), HILTONOL® (poly ICLC), polyadenylic-polyuridylic acid (poly A:U), RIBOXXIM® (RGIC®100), RIBOXXON® (RGIC®50 bioconjugate), and RIBOXXOL® (RGIC®50).

In some embodiments, a nucleic acid compound provided by the invention is combined with polyinosinic:polycytidylic acid (poly I:C). In some embodiments, the nucleic acid compound is combined with HILTONOL® (poly ICLC). In some embodiments, the nucleic acid compound is combined with polyadenylic-polyuridylic acid (poly A:U). In some embodiments, the nucleic acid compound is combined with RIBOXXIM® (RGIC®100). In some embodiments, the nucleic acid compound is combined with RIBOXXON® (RGIC®50 bioconjugate). In some embodiments, the nucleic acid compound is combined with RIBOXXOL® (RGIC®50).

In some embodiments, a nucleic acid compound provided by the invention is combined with a TLR7 agonist. A TLR7 agonist is an agonist that causes a signaling response through TLR7. Non-limiting examples of TLR7 agonists include single stranded RNA (ssRNA), loxoribine (a guanosine analogue derivatized at positions N7 and C8), imidazoquinoline compounds (e.g., imiquimod and resiquimod), or derivatives thereof. Further exemplary TLR7 agonists include, but are not limited to, GS-9620 (Vesatolimod), imiquimod (ALDARA™), and resiquimod (R-848).

In some embodiments, a nucleic acid compound provided by the invention is combined with GS-9620 (Vesatolimod). In some embodiments, the nucleic acid compound is combined with imiquimod (ALDARA™). In some embodiments, the nucleic acid compound is combined with resiquimod (R-848).

In some embodiments, a nucleic acid compound provided by the invention is combined with a TLR9 agonist. A TLR9 agonist is an agonist that causes a signaling response through TLR9. Exemplary TLR9 agonists include, but are not limited to, CpG oligodeoxynucleotides (GpG ODNs). In some embodiments, the CpG ODN is a Class A CpG ODN (CpG-A ODN), a Class B CpG ODN (CpG-B ODN), or a Class C CpG ODN (CpG-B ODN).

In some embodiments, a nucleic acid compound provided by the invention is combined with a CpG oligodeoxynucleotide (CpG ODN). In some embodiments, the CpG ODN is a Class A CpG ODN (CpG-A ODN). In some embodiments, the CpG ODN is a Class B CpG ODN (CpG-B ODN). In some embodiments, the CpG ODN is a Class C CpG ODN (CpG-C ODN).

### Other Combinations

In some embodiments, a nucleic acid compound, or pharmaceutical compositions thereof, provided by the invention is combined (e.g., administered in combination) with a VISTA antagonist, an adenosine A2AR antagonist, a B7-H3 antagonist, a B7-H4 antagonist, a BTLA antagonist, a CTLA-4 antagonist, an IDO antagonist, or a KIR antagonist.

In some embodiments, a nucleic acid compound, or pharmaceutical compositions thereof, provided by the invention is combined (e.g., administered in combination) with an agonist comprising a polypeptide (e.g., antibody, or antigen binding portion thereof) that specifically binds to CD137 (4-1BB).

In some embodiments, a nucleic acid compound, or pharmaceutical compositions thereof, provided by the invention is combined (e.g., administered in combination) with an agonist comprising a polypeptide (e.g., antibody, or antigen binding portion thereof) that specifically binds to CD134 (OX40).

A nucleic acid compound described herein can replace or augment a previously or currently administered therapy. For example, upon treating with a nucleic acid compound, administration of the one or more additional active agents can cease or diminish, e.g., be administered at lower levels or dosages. In some embodiments, administration of the previous therapy can be maintained. In some embodiments, a previous therapy will be maintained until the level of the nucleic acid compound reaches a level sufficient to provide a therapeutic effect. The two therapies can be administered in combination.

Monitoring a subject (e.g., a human patient) for an improvement in a cancer, as defined herein, means evaluating the subject for a change in a disease parameter, e.g., a reduction in tumor growth. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a cancer described herein.

In some embodiments, a nucleic acid compound described herein is administered to modulate a T-cell response in a patient, for example, by increasing T-cell activation and/or proliferation. Enhancement of T cell proliferation, IFN production and secretion, and/or the cytolytic activity of T cells may be beneficial to patients in need thereof to treat a disease or condition. Accordingly, in some embodiments, a nucleic acid compound of the present invention is administered to a patent in need thereof to induce or increase T-cell activation, enhance T cell proliferation, induce the production and/or secretion of IFN, and/or induce a cytolytic T cell response.

In some embodiments, a nucleic acid compound described herein, can be employed in methods of detection and/or quantification of human RIG-Is in a biological sample. Accordingly, a nucleic acid compound, as described herein, is used to diagnose, prognose, and/or determine progression of disease (e.g., cancer) in a patient.

In some embodiments, the nucleic acid compound described here can be used in combination with vaccines to boost their immunogenicity. Such vaccines include without limitations existing and emerging vaccines against infectious diseases such as Influenza virus, Respiratory syncytial virus, Rota virus, Ebola virus, Polio virus, Small pox virus, Cow pox virus, Monkey pox virus, MUMPs, Hepatitis virus B, and bacterial including Clostridiulm tetani, Mycobacterium tuberculosis, parasites such as Malaria etc.

TABLE of
Compounds (connector element (L) underlined)

| Cmpd # | Sequence |
|---|---|
| Cmpd 1 | 5′-ppGGAUCGAUCGAUCGUU(NH₂C₆CH₂OH) |
| | CGCGAUCGAUCGAUCC-3′ |
| Cmpd 2 | 5′-ppGGAUCGAUCGAUCGUU(NHC₆CH₂OH) |
| | (C6-biotin-NHS)CGCGAUCGAUCGAUCC-3′ |
| Cmpd 3 | 5′-ppGGAUCGAUCGAUCGUU(NHC₆CH₂OH) |
| | (IRD800)CGCGAUCGAUCGAUCC-3′ |
| Cmpd 4 | 5′-ppGGAUCGAUCGAUCGUU(NHC₆CH₂OH) |
| | (folic acid NHS)CGCGAUCGAUCGAUCC-3′ |
| Cmpd 5 | 5′-ppGGAUCGAUCGAUCGUU(NHC₆CH₂OH)(beta |
| | sitosterol)CGCGAUCGAUCGAUCC-3′ |
| Cmpd 6 | 5′-ppGGAUCGAUCGAUCGUU(NHC₆CH₂OH) |
| | (oleic acid)CGCGAUCGAUCGAUCC-3′ |

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, selected methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or 10%, more preferably 5%, even more preferably +1%, and still more preferably 0.1% from the specified value, as such variations are appropriate to perform the disclosed methods.

Agonist: As used herein, the term “agonist” is used in its broadest sense and encompasses any molecule or compound that partially or fully promotes, induces, increases, and/or activates a biological activity of a native polypeptide disclosed herein. Agonist molecules according to the disclosure may include nucleic acids (e.g., oligonucleotides, polynucleotides), antibodies or antigen-binding fragments, fragments or amino acid sequence variants of native polypeptides, peptides, oligonucleotides, lipids, carbohydrates, and small organic molecules. In some embodiments, activation in the presence of the agonist is observed in a dose-dependent manner. In some embodiments, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% higher than the signal measured with a negative control under comparable conditions. Also disclosed herein, are methods of identifying agonists suitable for use in the methods of the disclosure. For example, these methods include, but are not limited to, binding assays such as enzyme-linked immuno-absorbent assay (ELISA), Forte Bio© systems, fluorescence polarization (FP) assay, and radioimmunoassay (RIA). These assays determine the ability of an agonist to bind the polypeptide of interest (e.g., a receptor or ligand) and therefore indicate the ability of the agonist to promote, increase or activate the activity of the polypeptide. Efficacy of an agonist can also be determined using functional assays, such as the ability of an agonist to activate or promote the function of the polypeptide. For example, a functional assay may comprise contacting a polypeptide with a candidate agonist molecule and measuring a detectable change in one or more biological activities normally associated with the polypeptide. The potency of an agonist is usually defined by its EC₅₀ value (concentration required to activate 50% of the agonist response). The lower the EC₅₀ value the greater the potency of the agonist and the lower the concentration that is required to activate the maximum biological response.

Ameliorating: As used herein, the term “ameliorating” refers to any therapeutically beneficial result in the treatment of a disease state, e.g., cancer, including prophylaxis, lessening in the severity or progression, remission, or cure thereof.

Amino acid: As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

Amino acid substitution: As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g., insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

Base Composition: As used herein, the term “base composition” refers to the proportion of the total nucleotides of a nucleic acid (e.g., an RNA) consisting of guanine (or hypoxanthine)+cytosine and/or uracil (or thymine)+adenine nucleobases.

Base Pair: As used herein, the term “base pair” refers to two nucleobases on opposite complementary polynucleotide strands, or regions of the same strand, that interact via the formation of specific hydrogen bonds. As used herein, the term “Watson-Crick base pairing”, used interchangeably with “complementary base pairing”, refers to a set of base pairing rules, wherein a purine always binds with a pyrimidine such that the nucleobase adenine (A) forms a complementary base pair with thymine (T) and guanine (G) forms a complementary base pair with cytosine (C) in DNA molecules. In RNA molecules, thymine is replaced by uracil (U), which, similar to thymine (T), forms a complementary base pair with adenine (A). The complementary base pairs are bound together by hydrogen bonds and the number of hydrogen bonds differs between base pairs. As is known in the art, guanine (G)-cytosine (C) base pairs are bound by three (3) hydrogen bonds and adenine (A)-thymine (T) or uracil (U) base pairs are bound by two (2) hydrogen bonds.

Base-pairing interactions that do not follow these rules can occur in natural, non-natural, and synthetic nucleic acids and are referred to herein as “non-Watson-Crick base pairing” or alternatively “non-canonical base pairing” and universal nucleobases. A “wobble base pair” is a pairing between two nucleobases in RNA molecules that does not follow Watson-Crick base pair rules. For example, inosine is a nucleoside that is structurally similar to guanosine, but is missing the 2-amino group. Inosine is able to form two hydrogen bonds with each of the four natural nucleobases (Oda et al., (1991) Nucleic Acids Res 19:5263-5267) and it is often used by researchers as a “universal” base, meaning that it can base pair with all the naturally-occurring or canonical bases. The four main wobble base pairs are the guanine-uracil (G-U) base pair, the hypoxanthine-uracil (I-U) base pair, the hypoxanthine-adenine (I-A) base pair, and the hypoxanthine-cytosine (I-C) base pair. In order to maintain consistency of nucleic acid nomenclature, “I” is used for hypoxanthine because hypoxanthine is the nucleobase of inosine; nomenclature otherwise follows the names of nucleobases and their corresponding nucleosides (e.g., “G” for both guanine and guanosine as well as for deoxyguanosine). The thermodynamic stability of a wobble base pair is comparable to that of a Watson-Crick base pair. Wobble base pairs play a role in the formation of secondary structure in RNA molecules.

Biologically active: As used herein, the phrase “biologically active” refers to a characteristic of any substance that has activity in a biological system and/or organism. For instance, a substance that, when administered to an organism, has a biological effect on that organism, is considered to be biologically active and thus have “biological activity”. In particular embodiments, where a nucleic acid is biologically active, a portion of that nucleic acid that shares at least one biological activity of the whole nucleic acid is typically referred to as a “biologically active” portion.

Blunt-end: As used herein, the term “blunt-end” “blunt-ended” refers to the structure of an end of a duplexed or double-stranded nucleic acid, wherein both complementary strands comprising the duplex terminate, at least at one end, in a base pair. Hence, neither strand comprising the duplex extends further from the end than the other.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In certain embodiments, all nucleotide residues of the first portion are capable of base pairing with nucleotide residues in the second portion.

Covalently linked: As used herein, the term “covalently linked” (alternatively “conjugated”, “linked,” “attached,” “fused”, or “tethered”), when used with respect to two or more moieties, means that the moieties are physically associated or connected with one another, by whatever means including chemical conjugation, recombinant techniques or enzymatic activity, either directly or via one or more additional moieties that serves as a linking agent, to form a structure that is sufficiently stable so that the moieties remain physically associated under the conditions in which the structure is used, e.g., physiological conditions.

As used herein, the term “fragment,” as applied to a nucleic acid, refers to a subsequence of a larger nucleic acid. A “fragment” of a nucleic acid can be at least about 5 nucleotides in length; for example, at least about 10 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Homologous, homology” or “identical, identity” as used herein, refer to comparisons among amino acid and nucleic acid sequences. When referring to nucleic acid molecules, “homology,” “identity,” or “percent identical” refers to the percent of the nucleotides of the subject nucleic acid sequence that have been matched to identical nucleotides by a sequence analysis program. Homology can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the ExPaSy is used to align sequence fragments of genomic DNA sequences. However, equivalent alignment assessments can be obtained through the use of any standard alignment software.

As used herein, “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTG-3′ and 5′-AATC-3′ share 50% homology.

The term “hybridization” refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example, two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature and the concentration of salt in the reaction. Hybridizations are usually performed under stringent conditions, for example, at a salt concentration of no more than 1 M and a temperature of at least 25 °C. For example, conditions of 5×SSPE (750 mM NaCl, 50 mM Na Phosphate, 5 mM EDTA, pH 7.4) or 100 mM MES, 1 M NaCl, 20 mM EDTA, 0.01% Tween-20 and a temperature of 25-50 °C are suitable for allele-specific probe hybridizations. In a particularly preferred embodiment, hybridizations are performed at 40-50 °C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, Calif.).

A first oligonucleotide anneals with a second oligonucleotide with “high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 2012, Molecular Cloning: A Laboratory Manual, Cold Spring Harbor Laboratory Press, Cold Spring Harbor, N.Y.).

In need: As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a compound or composition comprising a RIG-I agonist).

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

As used herein, “isolate” refers to a nucleic acid obtained from an individual, or from a sample obtained from an individual. The nucleic acid may be analyzed at any time after it is obtained (e.g., before or after laboratory culture, before or after amplification).

The term “label” as used herein refers to a luminescent label, a light scattering label or a radioactive label. Fluorescent labels include, but are not limited to, the commercially available fluorescein phosphoramidites such as Fluoreprime (Pharmacia), Fluoredite (Millipore) and FAM (ABI). See U.S. Pat. No. 6,287,778.

The term “mismatch,” refers to a nucleic acid whose sequence is not perfectly complementary to a particular target sequence. The mismatch may comprise one or more bases. As used herein, the term “nucleic acid” refers to both naturally-occurring molecules such as DNA and RNA, but also various derivatives and analogs.

Modified: As used herein “modified” or “modification” refers to a changed state or change in structure resulting from a modification of a polynucleotide, e.g., RNA. Polynucleotides may be modified in various ways including chemically, structurally, and/or functionally. For example, the RNA molecules of the present disclosure may be modified by the incorporation of a non-natural base or a sequence motif, comprising a functional sequence or secondary structure, that provides a biological activity. In one embodiment, the RNA is modified by the introduction of non-natural or chemically-modified bases, nucleosides and/or nucleotides, e.g., as it relates to the natural ribonucleotides A, U, G, and C.

The term “nucleotide base,” as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6-methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N⁶-delta 2-isopentenyladenine (6iA), N⁶-delta 2-isopentenyl-2-methylthioadenine (2 ms6iA), N²-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, 0⁶-methylguanine, N⁶-methyladenine, 0⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT Application Publication WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C₁-C₆)alkoxyribose, 2′-(C₅-C₁₄)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C₁-C₆)alkylribose, 2′-deoxy-3′-(C₁-C₆)alkoxyribose and 2′-deoxy-3′-(C₅-C₁₄) aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-anomeric nucleotides, 1′-anomeric nucleotides, 2′-4′- and 3′-4′-linked and other “locked” or “LNA”, bicyclic sugar modifications (see, e.g., PCT Application Publications nos. WO 98/22489, WO 98/39352; and WO 99/14226). The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “overhang,” as used herein, refers to terminal non-base pairing nucleotide(s) resulting from one strand or region extending beyond the terminus of the complementary strand to which the first strand or region forms a duplex. One or more polynucleotides that are capable of forming a duplex through hydrogen bonding can have overhangs. The single-stranded region extending beyond the 3′-end and/or the 5′-end of the duplex is referred to as an overhang.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this disclosure are preferably those that result in the formation of stable or chemically feasible compounds.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙaryl (where n is 0-2), S(O)nheteroaryl (where n is 0-2), S(O)nheterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The term “halo” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S. The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain containing 2-8 carbon atoms and characterized in having one or more double bonds. Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The terms “alkylamino” and “dialkylamino” refer to —NH(alkyl) and —NH(alkyl)2 radicals respectively. The term “aralkylamino” refers to a —NH(aralkyl) radical. The term “alkoxy” refers to an —O-alkyl radical, and the terms “cycloalkoxy” and “aralkoxy” refer to an —O-cycloalkyl and O-aralkyl radicals respectively. The term “siloxy” refers to a R₃SiO— radical. The term “mercapto” refers to an SH radical. The term “thioalkoxy” refers to an —S-alkyl radical.

The term “alkylene” refers to a divalent alkyl (i.e., —R—), e.g., —CH₂—, —CH₂CH₂—, and —CH₂CH₂CH₂—. The term “alkylenedioxo” refers to a divalent species of the structure —O—R—O—, in which R represents an alkylene.

The term “aryl” refers to an aromatic monocyclic, bicyclic, or tricyclic hydrocarbon ring system, wherein any ring atom can be substituted. Examples of aryl moieties include, but are not limited to, phenyl, naphthyl, anthracenyl, and pyrenyl.

The term “cycloalkyl” as employed herein includes saturated cyclic, bicyclic, tricyclic, or polycyclic hydrocarbon groups having 3 to 12 carbons, wherein any ring atom can be substituted. The cycloalkyl groups herein described may also contain fused rings. Fused rings are rings that share a common carbon-carbon bond or a common carbon atom (e.g., spiro-fused rings). Examples of cycloalkyl moieties include, but are not limited to, cyclohexyl, adamantyl, and norbornyl, and decalin.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, piperidinyl, morpholino, pyrrolinyl and pyrrolidinyl.

The term “cycloalkenyl” as employed herein includes partially unsaturated, nonaromatic, cyclic, bicyclic, tricyclic, or polycyclic hydrocarbon groups having 5 to 12 carbons, preferably 5 to 8 carbons, wherein any ring atom can be substituted. The cycloalkenyl groups herein described may also contain fused rings. Fused rings are rings that share a common carbon-carbon bond or a common carbon atom (e.g., spiro-fused rings). Examples of cycloalkenyl moieties include, but are not limited to cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocycloalkenyl groups herein described may also contain fused rings. Fused rings are rings that share a common carbon-carbon bond or a common carbon atom (e.g., spiro-fused rings). Examples of heterocycloalkenyl include but are not limited to tetrahydropyridyl and dihydropyran.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heteroaryl groups herein described may also contain fused rings that share a common carbon-carbon bond.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

Patient: As used herein, the term “patient” includes human and other mammalian subjects that receive either prophylactic or therapeutic treatment.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length.

Pharmaceutically acceptable: As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

Pharmaceutically acceptable carrier: As used herein, the term “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

Phosphate: The term “phosphate” as used herein means a salt or ester of phosphoric acid. Polyphosphates are salts or esters of polymeric oxyanions formed from tetrahedral PO₄ (phosphate) structural units linked together by sharing oxygen atoms. As used herein, the term “diphosphate” refers to a polyphosphate comprising two phosphate structural units. As used herein, the term “triphosphate” refers to a polyphosphate comprising three phosphate structural units. In some embodiments, the disclosure provides a RIG-I agonist comprising a diphosphate moiety, or a derivative or analog thereof, linked to the 5′ terminus. In some embodiments, the disclosure provides a RIG-I agonist comprising a triphosphate moiety, or a derivative or analog thereof, linked to the 5′ terminus.

Preventing: As used herein, the term “preventing” when used in relation to a condition, refers to administration of a composition which reduces the frequency of, or delays the onset of, symptoms of a medical condition in a subject relative to a subject which does not receive the composition.

Purified: As used herein, the term “purified” or “isolated” as applied to any of the proteins (antibodies or fragments) described herein refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

RIG-I agonist: As used herein, the term “RIG-I agonist” refers to a nucleic acid compound (e.g., an RNA) that binds to RIG-I receptors and partially or fully promotes, induces, increases, and/or activates a biological activity, response, and/or downstream pathway(s) mediated by RIG-I signaling or other RIG-I-mediated function. Examples of RIG-I agonists are provided herein.

Subject: As used herein, the term “subject” includes any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

Therapeutically effective amount: As used herein, the terms “therapeutically effective amount” or “therapeutically effective dose,” or similar terms used herein are intended to mean an amount of an agent (e.g., a synthetic RIG-I agonist of the invention) that will elicit the desired biological or medical response, such as, for example, curing or at least partially arresting the condition or disease and its complications in a patient already suffering from the disease (e.g., an improvement in one or more symptoms of a cancer). Amounts effective for this use will depend on the severity of the disorder being treated and the general state of the patient's own immune system.

Treat: The terms “treat,” “treating,” and “treatment,” as used herein, refer to therapeutic or preventative measures described herein. The methods of “treatment” employ administration to a subject, in need of such treatment, a nucleic acid compound of the present disclosure, in order to prevent, cure, delay, reduce the severity of, or ameliorate one or more symptoms of the disorder or recurring disorder, or in order to prolong the survival of a subject beyond that expected in the absence of such treatment.

Conventional notation is used herein to describe polynucleotide sequences: the left-hand end of a single-stranded polynucleotide sequence is the 5′-end. The DNA strand having the same sequence as an mRNA is referred to as the “coding strand”; sequences on the DNA strand which are located 5′-to a reference point on the DNA are referred to as “upstream sequences”; sequences on the DNA strand which are 3′ to a reference point on the DNA are referred to as “downstream sequences.”

The skilled artisan will understand that all nucleic acid sequences set forth herein throughout in their forward orientation, are also useful in the compositions and methods of the invention in their reverse orientation, as well as in their forward and reverse complementary orientation, and are described herein as well as if they were explicitly set forth herein.

The term “ribonucleotide” and the phrase “ribonucleic acid” (RNA), as used herein, refer to a modified or unmodified nucleotide or polynucleotide comprising at least one ribonucleotide unit. A ribonucleotide unit comprises an oxygen attached to the 2′-position of a ribosyl moiety having a nitrogenous base attached in N-glycosidic linkage at the 1′-position of a ribosyl moiety, and a moiety that either allows for linkage to another nucleotide or precludes linkage.

The term “target” as used herein refers to a molecule that has an affinity for a given molecule. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, proteins, peptides, oligonucleotides and nucleic acids.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Synthesis of the Compounds:

The nucleic acid compounds of the invention can be synthesized by standard solid-phase synthesis. Various modifications can be introduced by methods known in the art. For example, site-specific phosphorothioate internucleotidic linkage (PS) is introduced during solid-phase assembly using any one of the sulfurizing reagents such as 3H-1,2-Benzodithiol-3-one 1,1-dioxide or 3-((Dimethylamino-methylidene)amino)-3H-1,2,4-dithiazole-3-thione, DDTT). The incorporation of the diphosphate or triphosphate groups in the oligonucleotides can be incorporated during solid-phase assembly by using protocols described in the literature.

For the synthesis of the amino-linked oligonucleotide of the invention, the corresponding phosphoramidite building block is incorporated during the oligonucleotide assembly during solid-phase assembly.

In one embodiment, an amino-modified oligonucleotide is prepared by automated solid-phase synthesis using standard phosphoramidite chemistry in conjunction with the corresponding nucleoside phosphoramidite building blocks. Thus, to the CPG-support-bound 2′-O-TBDMS protected N-benzoyl-cytosine nucleoside, the appropriate protected nucleoside phosphoramidite building blocks are sequentially added using a synthetic cycle involving detritylation, coupling, oxidation and capping. For incorporation of the amino-terminated moiety, the following modified phosphoramidite building block was employed.

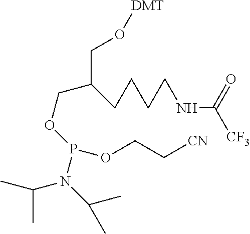

The oligonucleotide assembly was continued until the final G-nucleoside building block was added at the 5′-end. For the oxidation of each of the internucleotidic phosphite linkage, tert-butyl hydroperoxide was employed. Next, the support-bound oligo was diphosphorylated on the synthesizer using the fully automated procedure described by Zlatev et al., Solid-Phase Chemical Synthesis of 5′-Triphosphate DNA, RNA, and Chemically Modified Oligonucleotides. Current Protocols in Nucleic Acid Chemistry (2012) 1.28.1-1.28.16. In this protocol, the full-length 5′-hydroxyl oligonucleotides was initially converted to the corresponding 5′-H-phosphonate mono-esters by reaction with diphenyl phosphite in pyridine. Th intermediate H-phosphonate was then activated in the presence of imidazole and bromo-trichloromethane to the activated 5′-phosphorimidazolidates, and finally reacted with tributylammonium monophosphate to form the target diphosphate compound. The reagents for diphosphorylation procedure (1M diphenyl phosphite in pyridine, 0.1 M triethylammonium bicarbonate (TEAB) in water/acetonitrile, 1M imidazole/1M N,O-bis(trimethylsilyl)acetamide in CBrCl3/acetonitrile/triethylamine and 0.25M tributylammonium monophosphate in dimethylformamide/Acetonitrile) were prepared as described in Zlatev et al.

Cleavage and deprotection of the support-bound oligo. Following the completion of oligonucleotide assembly, the support-bound oligo in the column was treated with a mixture of AMA (1:1 mixture of methylamine (40% in water):ammonium hydroxide (28-30%, JT Baker) (enough to cover and moisten the entire support) and incubated at room temperature for 15 min. Then the solution was collected, and the procedure was repeated two more times. Finally, the column was washed with AMA into the same vial. The resulting solution was then incubated at 65 °C for 15 min with shaking to deprotect bases, then frozen at −80 °C for 1 h and dried in SpeedVac. Ethanol was added to the dried oligo and the material was dried in SpeedVac again to remove all traces of water.

2′-O-TBDMS deprotection. The removal of the 2′-OH protecting groups was carried out by treatment with 1M tetrabutylammonium fluoride (TBAF) in THE at room temperature for 36 h with shaking. It is important that the reaction mixture does not contain any traces of water, otherwise deprotection will be incomplete. Use of desiccator or a jar with desiccant is strongly recommended.

After incubation with TBAF, an equal volume of the 2M NaOAc was added to the mixture and the resulting solution was concentrated in SpeedVac to half the volume. Then the mixture was extracted three times with 1.6 volumes of EtOAc, evaporated in SpeedVac for 15 min to remove traces of EtOAc and precipitated with ethanol to get fully deprotected oligonucleotide. The deprotected oligos was further purified by RP-HPLC and ion-pair chromatography and the solution was lyophilized to obtain an off-while solid compound.

The amino functionalized oligonucleotide can then be employed for the synthesis of different conjugates as described herein and below. The compounds can be characterized by Mass spec, NMR, and other spectral methods.

### Synthesis of compound 1 (5′-ppGGAUCGAUCGAUCGUU—(NH₂C₆CH₂OH)CGCGAUCGAUCGAUCC-3′)

### Alternate Representation of Compound 1:

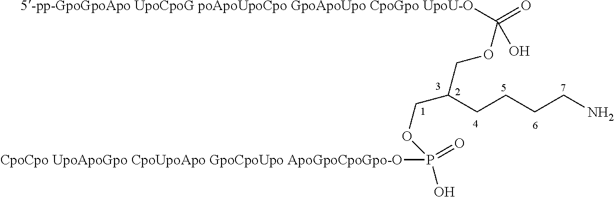

Compound 1 is assembled using standard phosphoramidite chemistry solid support. Compound 1 is assembled starting from the 3′-end of the oligonucleotide sequence. A trifunctional phosphoramidite reagent is used for the installation of the connector segment as shown in the Scheme 1 below and the oligonucleotide sequence is assembled until the last nucleotide at the 5′-end. Finally, the installation of diphosphoryl group (DP) is carried out via initial formation of 5′-H-phosphonate followed by reaction with tributylammonium monophosphate. Removal of the base and phosphate protecting groups by ammoniacal methyl amine is followed by NEt₃-HF to remove 2′-O-TBDMS. Purification steps include desalting, ion-exchange chromatography, ultrafiltration, and lyophilization. A second purification using RP-HPLC can be implemented.

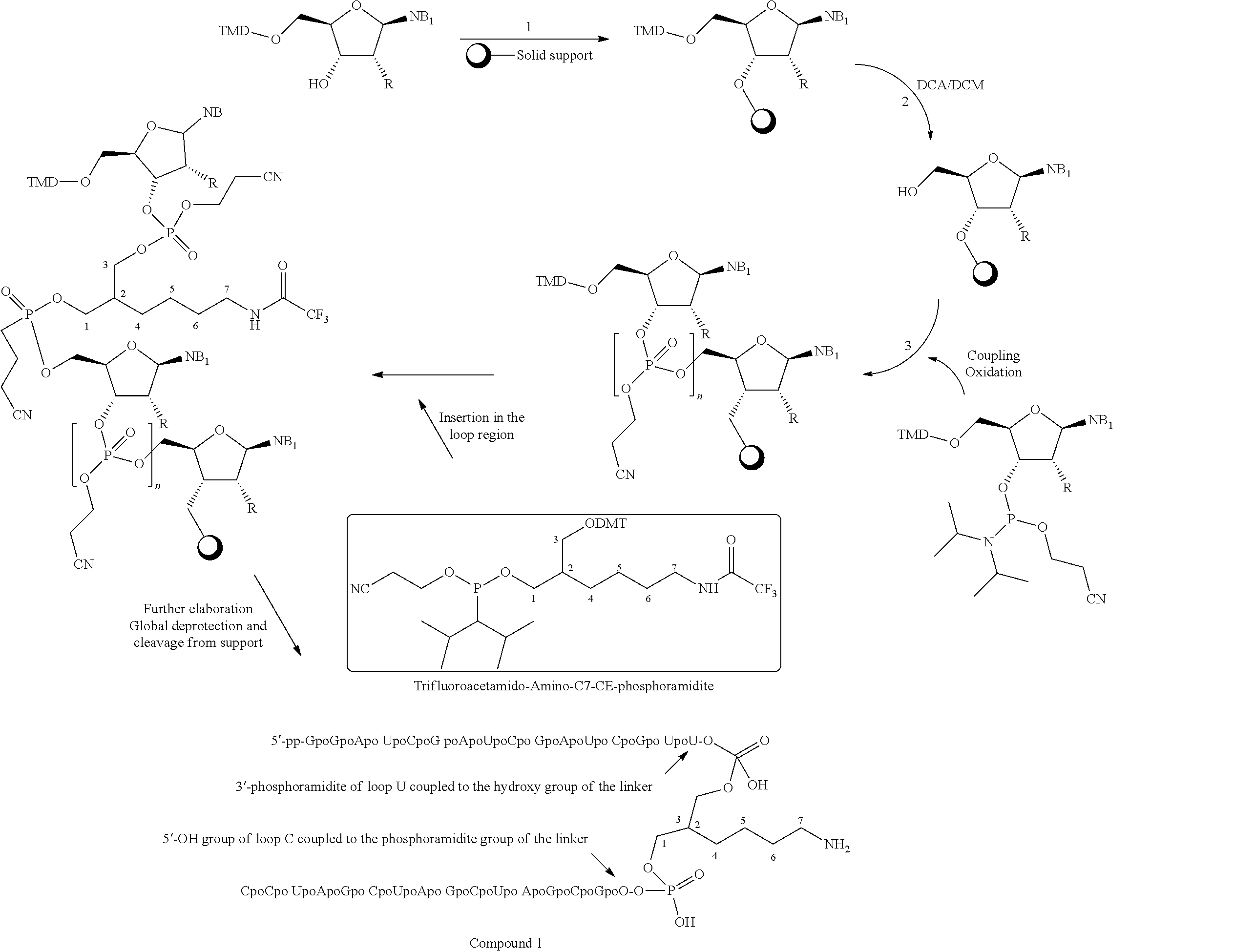

### Synthesis of a Biotin Conjugate (Compound 2):

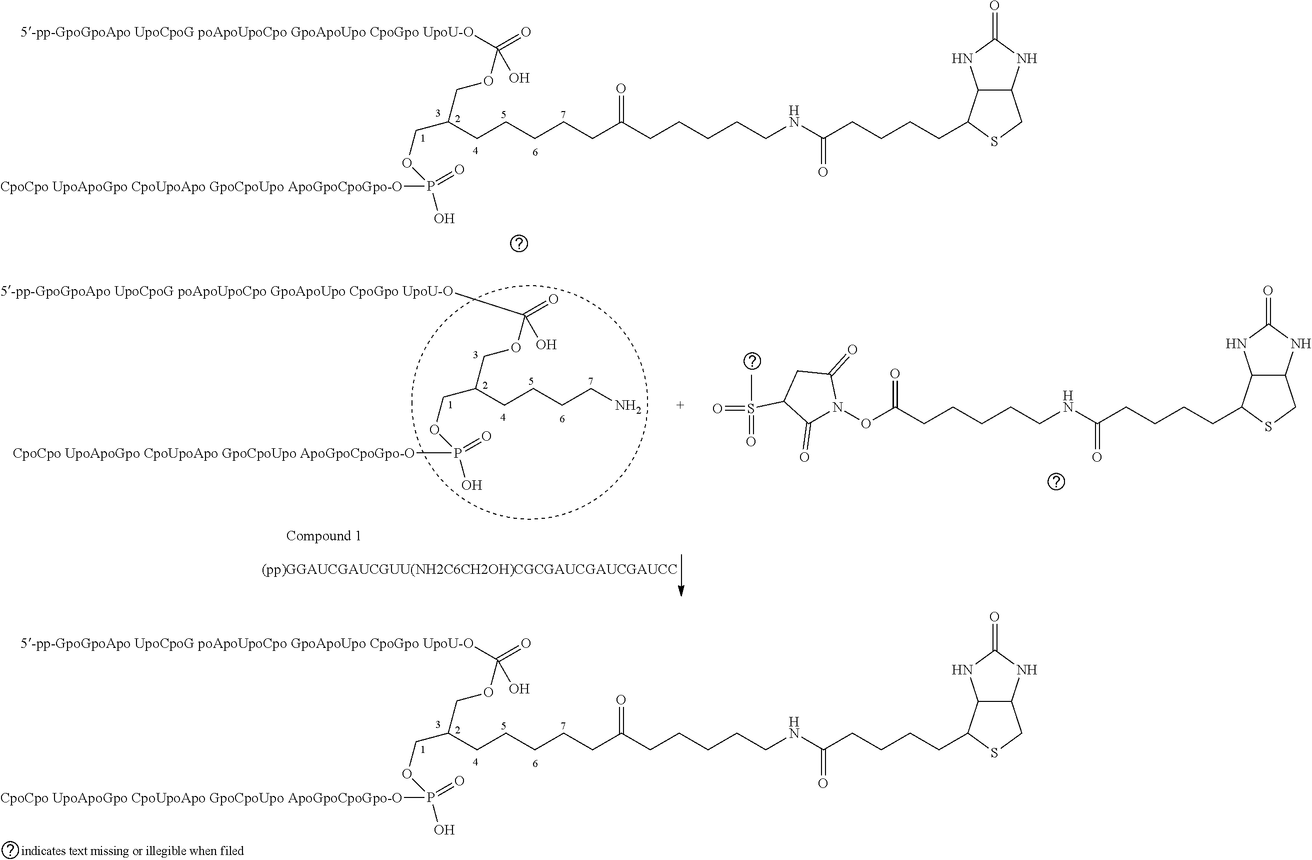

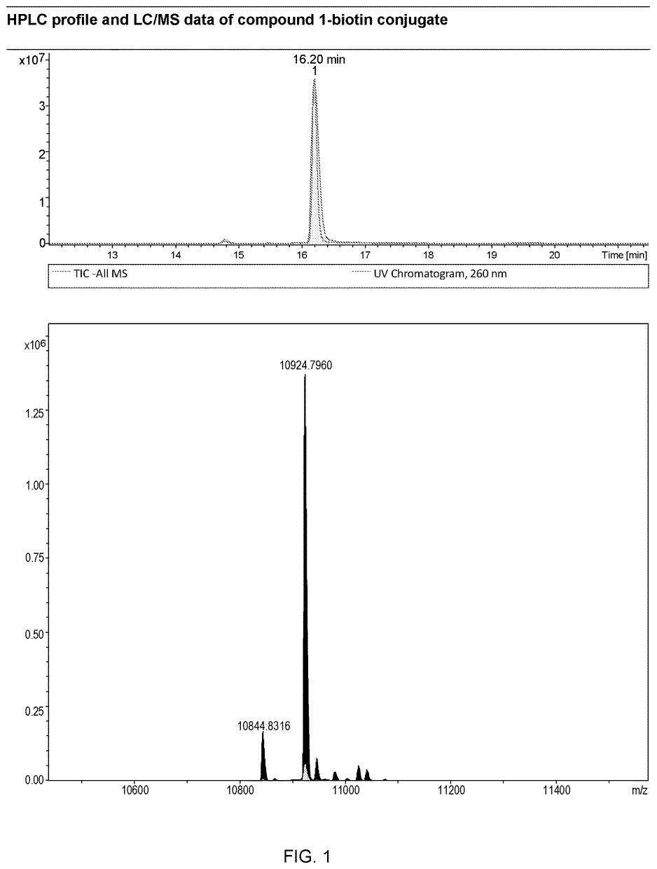

For the preparation of the oligo-biotin conjugate, the loop-modified oligo was reacted with activated ester of biotin (Sulfo-NHS-LC biotin) in aqueous acetonitrile or other solvents. Following the reaction, the crude conjugate was purified by preparative HPLC, desalted and lyophilized to get pure oligo-biotin conjugate. The HPLC and Mass spec data of the conjugate is shown in FIG. 1.

### Preparation of an IR-Dye Conjugate (Compound 3):

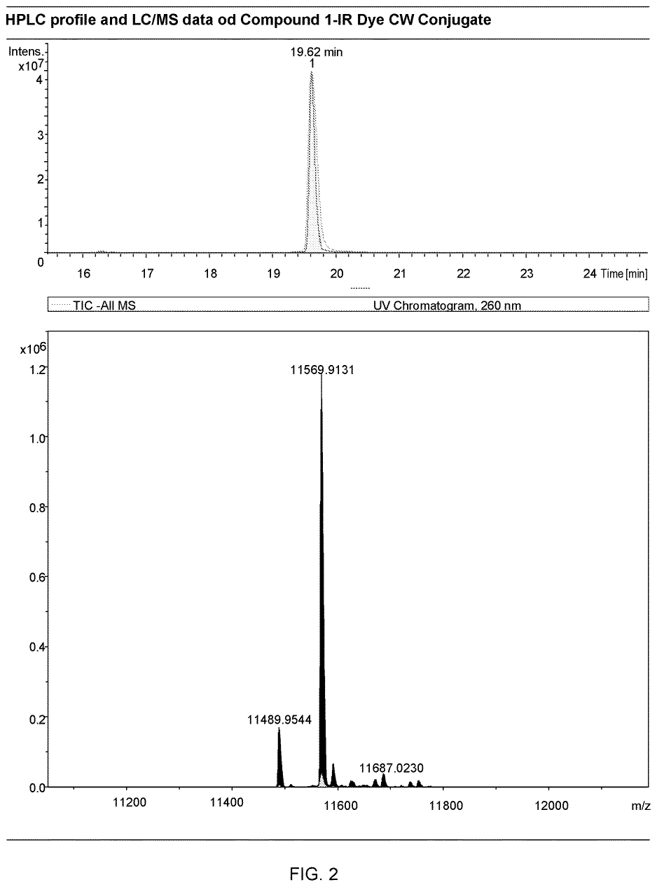

For the preparation of the oligo-dye conjugate, the loop-modified oligo was reacted with activated NHS ester of the dye in aqueous acetonitrile or other solvents. Following the reaction, the crude conjugate was purified by preparative HPLC, desalted and lyophilized to get pure oligo-biotin conjugate. The HPLC and Mass spec data of the conjugate is shown in FIG. 2.

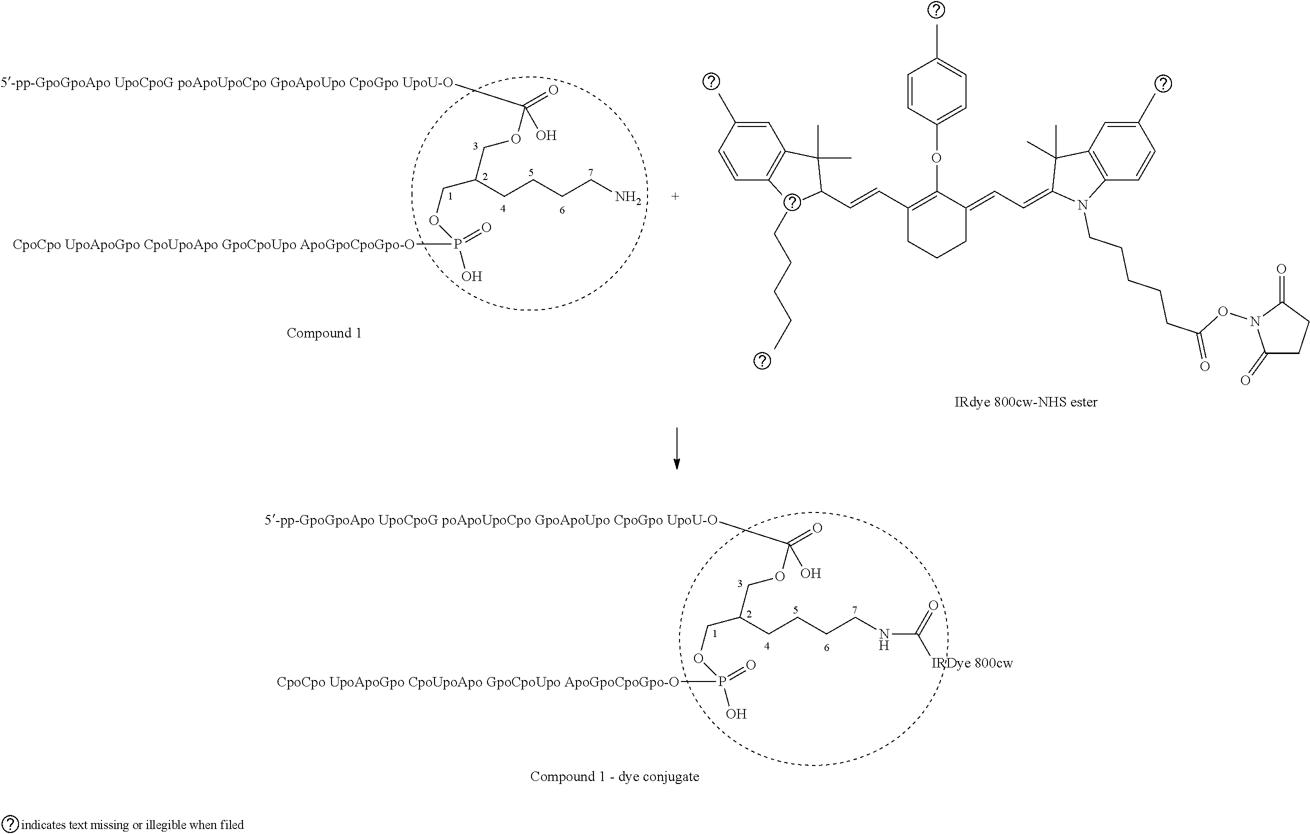

### Preparation of a Folic Acid Conjugate (Compound 4)

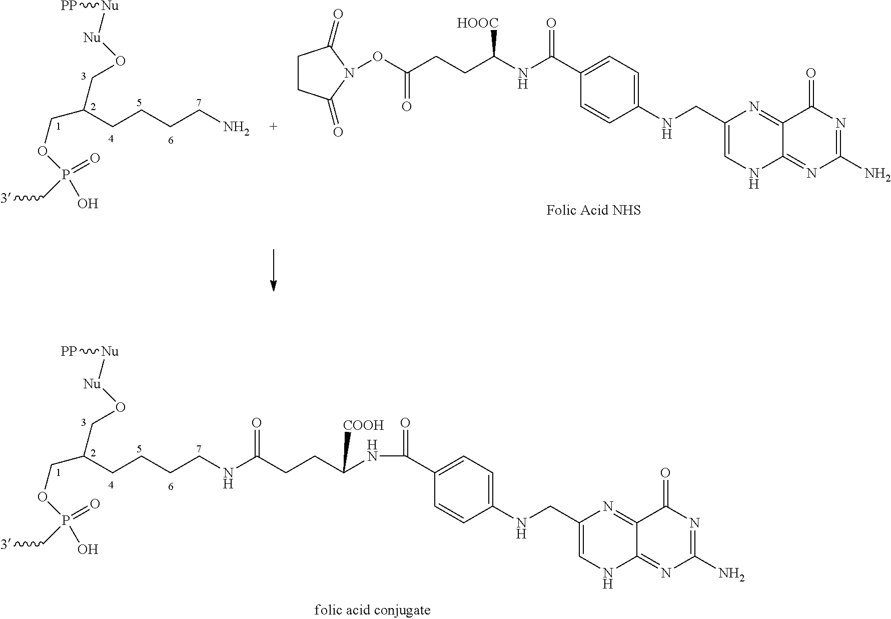

Compound 1 (10 micromol) is treated with the Folic acid N-hydroxy succinimide ester (Folic acid NHIS) in DMF solvent and stirred for 24 h at room temp. Work up and purification by HPLC chromatography gave the folic acid conjugate Compound 4.

### Preparation of a Beta Sitosterol Conjugate (Compound 5)

### Step 1

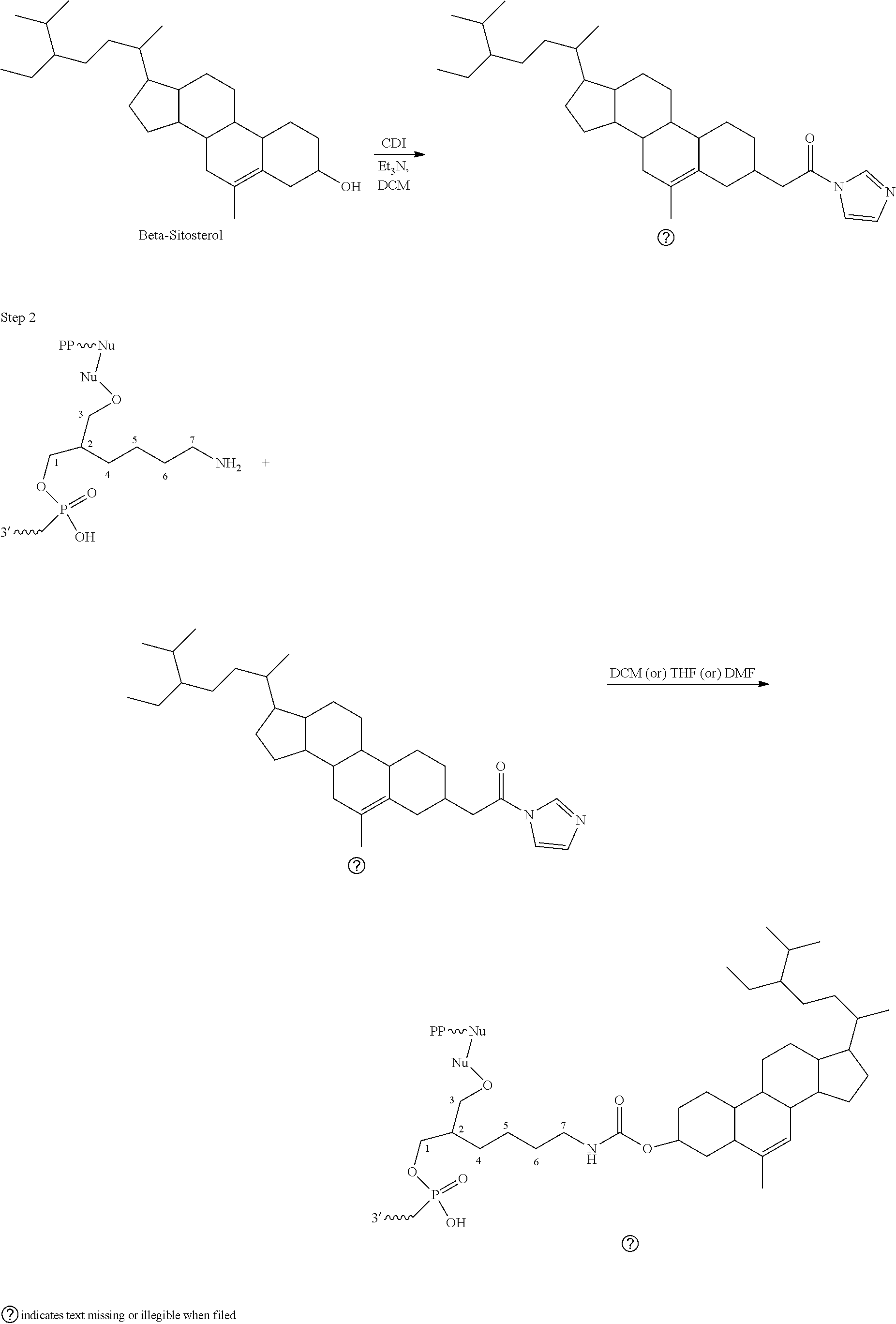

Compound 1 (10 micromol) is treated with the activated carbonyl imidazolide ester of 3-hydroxy-betasitosterol in DMF solvent and stirred for 24 h at room temp. Work up and purification by HPLC chromatography gave the beta sitosterol conjugate 5.

### Preparation of Oleic Acid Conjugate (Compound 6)

### Step 1

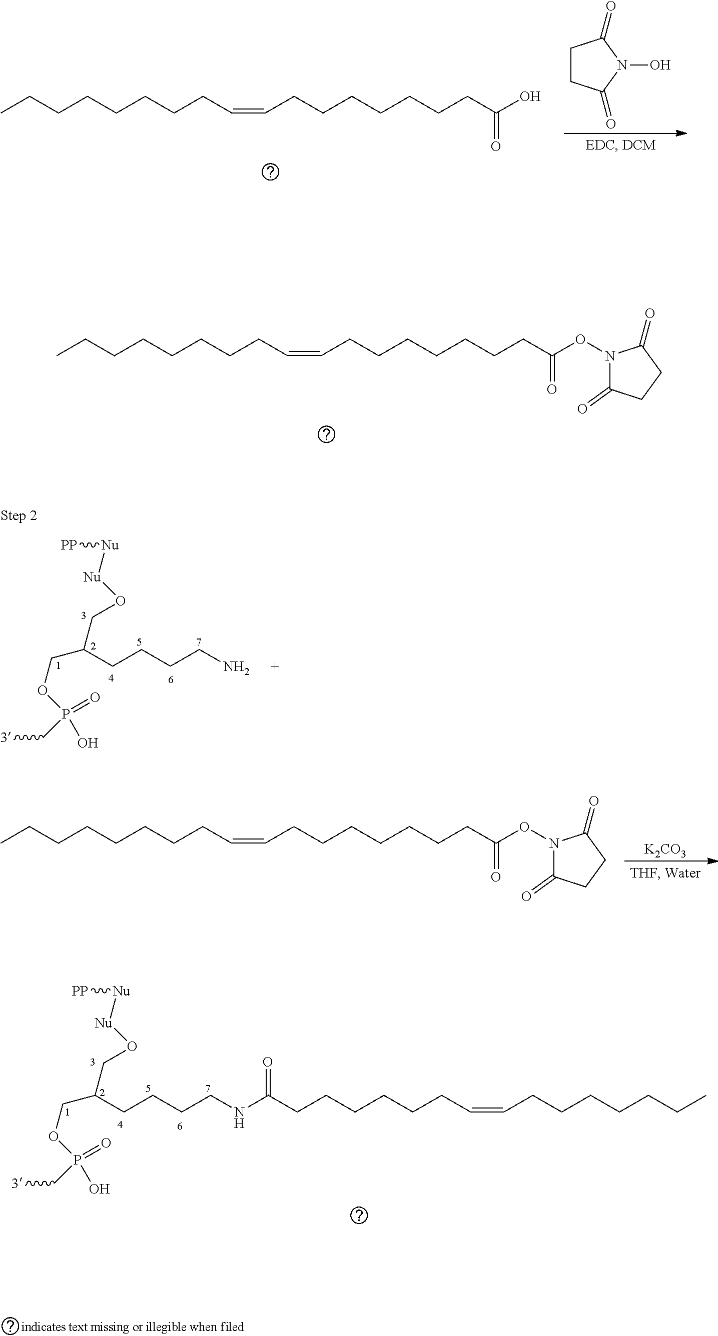

The N-hydroxy succinimide ester of oleic acid (1.5 mmol) is treated with the Compound 1 9 1 millimol) in the presence of 10% aqueous potassium carbonate. The contents are stirred for 24 to 48 hours until reaction is complete and no starting material (oligo compound) is present. The reaction mixture is subjected to organic extraction to remove the oleic acid intermediates and the resulting Oleic acid conjugate Compound 6 is obtained by RP—HPLC purification and lyophilization.

### RIG-I Agonist Activity of a Compound of the Invention

RIG-I and MDA-5 stimulation is tested by assessing IRF3 activation in HEK293 cells expressing human RIG-I or MDA-5 genes. The test articles include:

5′-pp GGAUCGAUCGAUCGUUCGCGAUCGAUCGAUCC-3′
(Compound X);
5′-(pp)GGAUCGAUCGAUCGUU(NH₂C₆CH₂OH)CGCGA
UCGAUCGAUCC-3′
(Compound 1);
poly (I:C);
5′-pppdsRNA positive control;
and
IFNα.

The activity of the test articles is tested on human RIG-I and MDA-5 expressing cells as potential agonists. The test articles are evaluated at one concentration on MDA-5 expressing cells and compared to control ligands. In addition, the activity of the test articles is evaluated at five concentrations on RIG-I expressing cells.

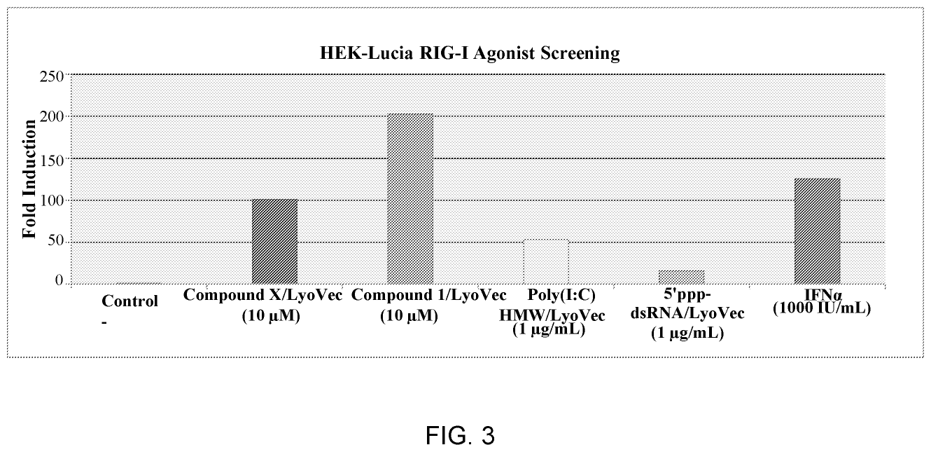

The secreted luciferase reporter is under the control of a promoter inducible by TRF transcription factors. This reporter gene allows the monitoring of signaling through the RIG-I and MDA-5 genes, based on the activation of IRF3. In a 96-well plate (200 pL total volume) containing the appropriate cells, 20 μL of the test article or of the positive control ligand is added to the wells. After a 16-24-hour incubation, activation of the IRF pathway is monitored using a luciferase detection assay. Luciferase activity is assayed from the supernatant of the induced cells, and the relative luminescence units (RLUs) are detected by a Promega GloMax Luminometer. FIG. 3 provides the results as relative luminescence units (RLUs). The RIG compounds (i.e., Compound X and Compound 1) did not activate MDA-5 (data not shown).

IRF stimulation can be tested by assessing activation in THP1-Dual cells, a human monocytic cell line that naturally expresses many pattern-recognition receptors (PRR). The compound of the invention can be evaluated at one concentration or several concentrations and compared to control ligands. This step can be performed in triplicate. The results can be provided as relative luminescence units (RLUs).

IRF stimulation can be tested by assessing activation in A549 cells, a lung epithelial cell line that naturally expresses many pattern-recognition receptors (PRR). The compound of the invention can be evaluated at one concentration or several concentrations and compared to control ligands. This step can be performed in triplicate. The results can be provided as relative luminescence units (RLUs).

### Selectivity Assays:

To evaluate the selectivity of the compounds of the invention as RIG-I agonist, the compounds can be evaluated in multiple cell lines expressing Toll-Like Receptor (TLR), NOD-Like Receptor (NLR) stimulation. The testing can be done by assessing NF-κB activation in HEK293 cells expressing a given TLR or NLR. The activity of the compound of the invention can be tested on seven human TLRs (TLR2, 3, 4, 5, 7, 8 and 9), two human NLRs (NOD1 and NOD2), eight mouse TLRs (2, 3, 4, 5, 7, 8, 9 and 13) and two mouse NLRs (NOD1 and NOD2) as potential agonists. The activity of the compound of the invention can be tested at one concentration and compared to control ligands. These steps can be performed in triplicate.

STING stimulation can be tested by assessing activation in THP1-Dual cells, a human monocytic cell line that naturally expresses many pattern-recognition receptors (PRR) including human STING. STING stimulation in THP1-Dual cells can be tested by assessing IRF activation. The compound of the invention can be evaluated at one concentration or in multiple concentrations and compared to control ligands. This step can be performed in triplicate. The results can be provided as relative luminescence units (RLUs).

### Evaluation of RIG-I Mediated Apoptosis

The compounds of the invention can be evaluated for cytokine/chemokine production in tumor cell lines, myeloid cell lines, and/or primary immune cells (e.g., PBMC, bone marrow-derived macrophages, and/or TILs) from mice and humans via Luminex, and cell death can be measured by CytoTox-Glo™ and/or Caspase/Annexin V/ICD markers by flow cytometry or Incucyte imaging. Different transfection reagents (e.g., Lipofectamine® RNAiMAX and JET-PEI) can be used to facilitate delivery of the compounds to the cytosol. Studies can be carried out using RIG-I knockout cells or through gene silencing using siRNAs or shRNAs directed against RIG-I to show functional dependence. Pro-inflammatory cytokine/chemokine detection can include NF-κB pathway readout (e.g., via NFKB reporter and IL6, TNFα ELISA).

### Antiviral Assays:

The compounds can be tested for antiviral activity against virus such as human rhinovirus, parainfluenza, influenza, coronavirus, or RSV using the appropriate cell lines such as HeLa, MDCK, Vero76 and A549 cells. The antiviral activity can be assessed at different concentrations ranging from 0.01 to 10 μg/mL using cytopathic assays and cytotoxicity can be assessed by Neutral Red assays. The EC₅₀ and CC₅₀ values can be assessed, and selectivity index can be calculated using CC₅₀/EC₅₀ ratios.

### Dose-Response of RIG-I Activation

### IRF Signalling Detection in A549 Dual Cell Assay System

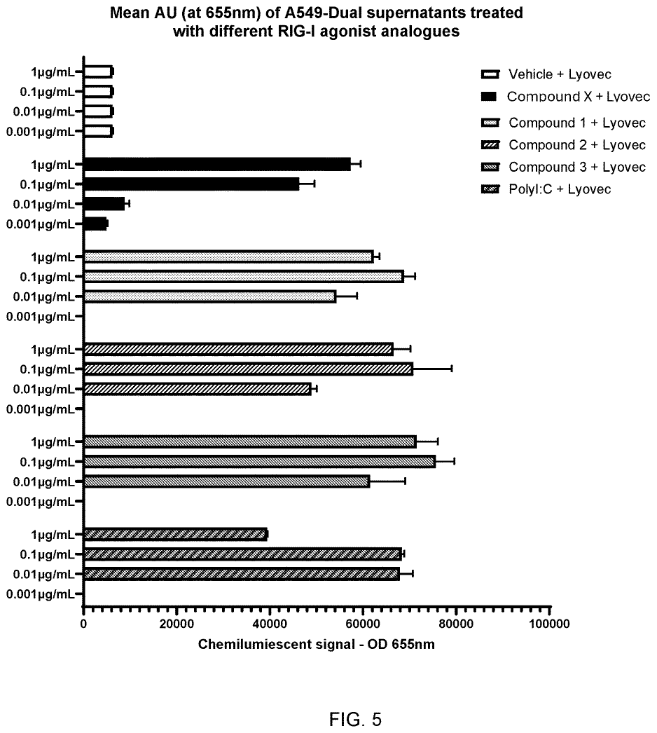

To determine the ability of the compound to activate interferon regulatory factor (IRF) signalling, A549-Dual™ cells (InvivoGen, Toulouse, France), expressing the Lucia luciferase gene, which encodes a secreted luciferase, under the control of an ISG54 minimal promoter in conjuiction with five IFN-stimulated response elements, were used. Firstly, A549-Dual™ cells were collected and resuspended at 2.8×10⁵ cells/ml in fresh, pre-warmed growth medium. 20 μL of a mixture of compound and reconstituted LyoVec (InvivoGen) was applied to 180 μL of cell suspension in the 96 well plate. After 18-24 hrs incubation at 37 °C in a CO₂ incubator, 20 μL of the supernatant was transferred to a 96-well white (Opaque) plate and the Luminescent reporter signal as a marker of activation of the IRF pathway, was detected in a multi-mode plate reader (CLARIOstar®, BMG LABTECH, Aylesbury, UK) after mixture with 50 μL of QUANTIU-Luc™ 4 reagent. As an assay control, 5′ppp-dsRNA (InvivoGen) was also used. Results are shown in FIG. 5.

### Induction of CXCL-10

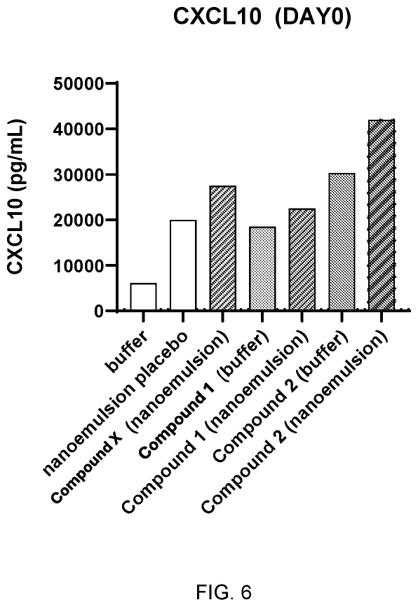

An air-liquid interphase (ALI) system is set up with cultures of lung epithelial cells which endogenously express RIG-I whose activation by RIG-I ligands results in induction of Interferon and interferon-stimulating genes such as CXCL10. The production of CXCL10 can be measured by ELISA. Treatment of ALI cultures with Compound 1 and conjugate compounds for 30 minutes and harvesting the cultures after 24 h exposure and performance of ELISA on the supernatant, gave good induction of CXCL10. As shown in FIG. 6, although compounds 1 and 2 showed good induction of CXCL10 as aqueous solutions compared to untreated controls, addition of a surfactant like polysorbate to the aqueous solution seem to enhance the uptake of the compounds and induction of CXCL10. The induction of CXCL10 is a clear indication that the tested compounds were capable of activating RIG-I and cause induction IFN signaling cascade.

1. A nucleic acid compound capable of inducing interferon production comprising a first nucleic acid sequence and a second nucleic acid sequence, wherein the first nucleic acid sequence and the second nucleic acid sequence are complementary to each other and hybridize to form a double-stranded section, wherein the number of base pairs in the double stranded section is an integer ranging from 8 to 19; and wherein the 3′ end for the first nucleotide sequence is conjugated to one end of a connector element and wherein the other end of the connector element is conjugated to the 5′ end of the second nucleotide sequence; and wherein the 5′ most nucleotide of the first nucleic acid sequence comprises a 5′ diphosphate or triphosphate moiety, or derivative or analog thereof.

2. The nucleic acid compound according to claim 1, wherein the nucleic acid compound has the structure of Formula I,
5′-P_{z}-(N)_{b}N-3′-(E)_{y}(E)-L-(E)(E)_{y′}-5′-N(N)_{b′}-3′
Formula I
wherein
5′-P_{z}—(N)_{b}N-3′ represents the first nucleic acid sequence;
5′-N(N)_{b′}-3′ represents the second nucleic acid sequence;
P at each instance is independently a phosphate or analog thereof,
z is 2 or 3;
N is, at each instance, any nucleotide or modified nucleotide or analog or derivative there of;
b and b′ are independently 7, 8, 9, 10, 11, 12, 13, 14, 15, 16, 17, or 18;
5′-(E)_{y}(E)-L-(E)(E)_{y′}-3′ represents the connector element wherein E at each occurrence is independently any nucleotide, modified nucleotide, or abasic;
y and y′ are independently 0-9, provided that y+y′ equals 0-8;
L is a non-nucleotide segment having the structure 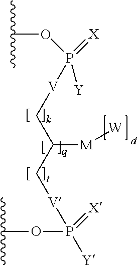
wherein
X and X′ are independently O or S;
Y and Y′ are independently OR″, SR″, or NRR′;
V and V′ are independently O, S, or NRR′;
q is 1-20;
k is 1-20;
t is 1-20;
M selected from aliphatic, substituted aliphatic, aryl, substituted aryl, heteroalkyl, heterocyclyl or substituted heterocyclyl;
W is any reactive group or conjugation group; and
d is 0 or 1.

3. The nucleic acid compound according to claim 2, wherein z is 2.

4. The nucleic acid compound according to claim 3, wherein at least one P is a phosphate analog.

5. The nucleic acid compound according to claim 3, wherein both P are phosphate analogs.

6. The nucleic acid compound according to claim 2, wherein z is 3.

7. The nucleic acid compound according to claim 6, wherein at least one P is a phosphate analog.

8. The nucleic acid compound according to claim 6, wherein at least two P are phosphate analogs.

9. The nucleic acid compound according to claim 6, wherein all 3 P are phosphate analogs.

10. The nucleic acid compound according to any one of claims 1-9, wherein the phosphate analog, when present, comprises the structure 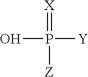
wherein
Y is O or S, or CH—R where R=alkyl, ar-alkyl, heteroaryl, cycloalkylamines (e.g., piperazines),
X is O or S, and
Z is OH, SH, NHR′, wherein R′ is H, alkyl, aralkyl, and heteroaryl.

11. The nucleic acid compound according to claim 1, wherein the nucleotides of the first and second nucleotide sequence are ribonucleic acids (RNA).

12. The nucleic acid compound according to claim 1, wherein b and b′ are independently 9, 10, 11, 12, 13, 14, 15, 16, 17, or 18; preferably 11, 12, 13, 14, 15, 16, 17, or 18; preferably 13, 14, 15, 16, 17, or 18.

13. The nucleic acid compound according to claim 1, wherein the compound is selected from
(SEQ ID NO: 1 and SEQ ID NO: 2)
5′-ppGGAUCGAUCGAUCGUU(NH₂C₆CH₂OH)
CGCGAUCGAUCGAUCC-3′;
(SEQ ID NO: 1 and SEQ ID NO: 2)
5′-ppGGAUCGAUCGAUCGUU(NHC₆CH₂OH)
(C6-biotin-NHS)CGCGAUCGAUCGAUCC-3′;
(SEQ ID NO: 1 and SEQ ID NO: 2)
5′-ppGGAUCGAUCGAUCGUU(NHC₆CH₂OH)
(IRD800)CGCGAUCGAUCGAUCC-3′;
(SEQ ID NO: 1 and SEQ ID NO: 2);
5′-ppGGAUCGAUCGAUCGUU(NHC₆CH₂OH)
(folic acid NHS)CGCGAUCGAUCGAUCC-3′
(SEQ ID NO: 1 and SEQ ID NO: 2)
5′-ppGGAUCGAUCGAUCGUU(NHC₆CH₂OH)
(beta sitosterol)CGCGAUCGAUCGAUCC-3′;
or
(SEQ ID NO: 1 and SEQ ID NO: 2)
5′-ppGGAUCGAUCGAUCGUU(NHC₆CH₂OH)
(oleic acid) CGCGAUCGAUCGAUCC-3′.

14. The nucleic acid compound according to claim 13, wherein the compound is
(SEQ ID NO: 1 and SEQ ID NO: 2)
5′-ppGGAUCGAUCGAUCGUU(NH₂C₆CH₂OH)
CGCGAUCGAUCGAUCC-3′.

15. A pharmaceutical composition comprising a nucleic acid compound according to claim 1 and a pharmaceutically acceptable excipient.

16. (canceled)

17. (canceled)

18. A method for the treatment of a disease or condition which is treated by stimulation or activation of the innate and/or adaptive immune system and/or the raising of an innate and/or adaptive immune response, comprising administering to a subject in need thereof a therapeutically or prophylactically effective amount of the nucleic acid compound according to claim 1 or a composition thereof.

19. The method according to claim 18, wherein the disease or condition is infection by a virus or associated with infection with such a virus.

20. The use or method according to claim 19, wherein the virus infects the respiratory tract and the disease associated with infection is a disease of the respiratory tract.

21. The method according to claim 18, wherein the disease or condition is cancer.
